(19) United States
(12) Patent Application Publication
(10) Pub. No.: US 2010/0063035 A1
(43) Pub. Date: Mar. 11, 2010

(54) CARBONIC ANHYDRASE INHIBITORS DERIVATIVES

(75) Inventors: Francesca Benedini, San Donato Milanese (Milano) (IT); Stefano Biondi, Pero (Milano) (IT); Ennio Ongini, Segrate (Milano) (IT)

Correspondence Address: ARENT FOX LLP, 1050 CONNECTICUT AVENUE, N.W., SUITE 400, WASHINGTON, DC, 20036, US

(73) Assignee: Nicox S.A., Sophia Antipolis Valbonne (FR)

(21) Appl. No.: 12/516,460

(22) PCT Filed: Dec. 3, 2007
(86) PCT No.: PCT/IB2007/003856

§ 371 Date: May 27, 2009

Related U.S. Application Data
(60) Provisional application No. 60/870,285, filed on Dec. 15, 2006.

(51) Int. Cl. A61K 31/542; A61K 31/382; C07D 513/02; C07D 335/04; A61P 9/12; A61P 27/00; A61P 3/10

(52) U.S. Cl. 514/226.5; 514/432; 544/48; 549/23

(57) ABSTRACT

Nitroderivatives of dorzolamide and brinzolamide having improved pharmacological activity and enhanced tolerability are described. They can be employed for the treatment of glaucoma, ocular hypertension, age-related macular degeneration, diabetic macular edema, diabetic retinopathy, hypertensive retinopathy and retinal vasculopathies.

The present invention relates to new carbonic anhydrase inhibitors derivatives. More particularly, the present invention relates to nitrooxyderivatives of dorzolamide and brinzolamide, pharmaceutical compositions containing them and their use as drugs for treating glaucoma, ocular hypertension, age-related macular degeneration, diabetic macular edema, diabetic retinopathy, hypertensive retinopathy and retinal vasculopathies.

Glaucoma is optic nerve damage, often associated with increased intraocular pressure (IOP), that leads to progressive, irreversible loss of vision.

Almost 3 million people in the United States and 14 million people worldwide have glaucoma; this is the third leading cause of blindness worldwide.

Glaucoma occurs when an imbalance in production and drainage of fluid in the eye (aqueous humor) increases eye pressure to unhealthy levels.

It is known that elevated IOP can be at least partially controlled by administering drugs which either reduce the production of aqueous humor within the eye or increase the fluid drainage, such as beta-adrenergic antagonists, α-adrenergic agonists, cholinergic agents, prostaglandin analogs or carbonic anhydrase inhibitors.

Several side effects are associated with the drugs conventionally used to treat glaucoma.

Topical beta-adrenergic antagonists show serious pulmonary side effects, depression, fatigue, confusion, impotence, hair loss, heart failure and bradycardia.

Topical α-adrenergic agonists have a fairly high incidence of allergic or toxic reactions; topical cholinergic agents (miotics) can cause visual side effects.

The topical prostaglandin analogs (bimatoprost, latanoprost, travoprost and unoprostone) used in the treatment of glaucoma, can produce ocular side effects, such as increased pigmentation of the iris, ocular irritation, conjunctival hyperaemia, iritis, uveitis and macular oedema (Martindale, Thirty-third edition, p. 1445).

Finally, the side effects associated with oral carbonic anhydrase inhibitors include fatigue, anorexia, depression, paresthesias and serum electrolyte abnormalities (The Merck Manual of Diagnosis and Therapy, Seventeenth Edition, M. H. Beers and R. Berkow Editors, Sec. 8, Ch. 100).

WO 2006/052899 discloses novel nitrosated and/or nitrosylated compounds or pharmaceutically acceptable salts thereof, and novel compositions, for treating ophthalmic disorders comprising at least one nitrosated and/or nitrosylated compound, and, optionally, at least one nitric oxide donor and/or at least one therapeutic agent selected from the group consisting of an α-adrenergic receptor agonist, an ACE inhibitor, an antimicrobial, a β-adrenergic antagonist, a carbonic anhydrase inhibitor, a non-steroidal anti-inflammatory drug, a prostaglandin, a COX-2 inhibitor and a steroid.

It is the object of the present invention to provide new derivatives of carbonic anydrase inhibitors able not only to eliminate or at least reduce the side effects associated with the parent compounds, but also to improve pharmacological activity. It has been surprisingly found that nitrooxyderivatives of carbonic anydrase inhibitors have a significantly improved overall profile as compared to native carbonic anydrase inhibitors both in terms of wider pharmacological activity, enhanced tolerability and long-acting ocular hypotensive activity. In particular, it has been recognized that the carbonic anydrase inhibitors nitroderivatives of the present invention can be employed for treating ocular hypertension and preventing glaucoma. Moreover, they have been found to be effective for the treatment of age-related macular degeneration, diabetic macular edema, diabetic retinopathy, hypertensive retinopathy and retinal vasculopathies.

The compounds of the present invention are indicated for the reduction of intraocular pressure in patients with open-angle glaucoma or with chronic angle-closure glaucoma who underwent peripheral iridotomy or laser iridoplasty.

An object of the present invention is a method for treating eye disorders, in particular glaucoma, ocular hypertension, age-related macular degeneration, diabetic macular edema, diabetic retinopathy, hypertensive retinopathy and retinal vasculopathies in a patient in need thereof comprising administering a therapeutically effective amount of a carbonic anhydrase inhibitor able to release nitric oxide.

A carbonic anhydrase inhibitor is a compound having an inhibition constant (K) against the isoenzyme CAII in the range of 0.01-200 nM. The carbonic anhydrase activity is measured according to the test on carbonic anhydrase inhibition as reported below.

A Carbonic Anhydrase Inhibitor able to release nitric oxide is a compound having an EC₅₀ value in the range of 1-50 μM, in vasorelaxation. The vasorelaxation is measured according to the test on vascular tone as reported below.

More particularly, object of the present invention is nitroderivatives of dorzolamide and brinzolamide of general formula (I) and pharmaceutically acceptable salts or stereoisomers thereof.

R—(X—Y—ONO₂)ₘ (I)

wherein:
m is an integer equal to 1 or 2, preferably m is 1;

### R is:

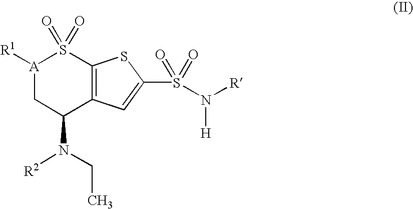

wherein

### R¹ is —CH₃ or —(CH₂)₃—OCH₃;

R² is H or a group —(X—Y—ONO₂);
R′ is H or a group —(X—Y—ONO₂);
with the proviso that at least one of R² or R′ is a —(X—Y—ONO₂) group;
A is a carbon or nitrogen atom;
preferably in formula (II) R¹ is —CH₃ and A is a carbon atom;

### X is —CO—, —COO—;

Y is a bivalent radical having the following meaning:
a)
straight or branched C₁-C₂₀ alkylene, preferably a straight or branched C₁-C₁₀ alkylene;
straight or branched C₁-C₂₀ alkylene substituted with one or more of the substituents selected from the group consisting of halogen atoms, hydroxy, —ONO₂ or T, wherein T is —OC(O)(C₁-C₁₀ alkyl)-ONO₂ or —O(C₁-C₁₀ alkyl)-ONO₂, preferably —ONO₂, preferably a straight or branched C₁-C₁₀ alkylene substituted with one or more —ONO₂;
   cycloalkylene with 5 to 7 carbon atoms into cycloalkylene ring, the ring being optionally substituted with side chains T₁, wherein T₁ is straight or branched C₁-C₁₀ alkyl;

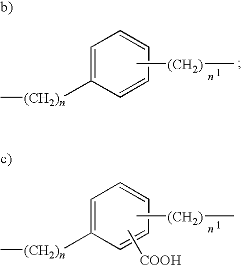

wherein n is an integer from 0 to 20, and n¹ is an integer from 1 to 20, preferably n is an integer from 0 to 5, preferably n¹ is an integer from 1 to 10 or from 1 to 5;

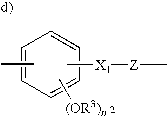

wherein
X₁=—OCO— or —COO—, preferably X₁ is —COO—;
Z is —(CH₂)ₙ¹— or the bivalent radical defined above under b), preferably Z is —(CH₂)ₙ¹—, wherein
n¹ is as defined above, preferably n¹ is an integer from 1 to 10;
n² is an integer from 0 to 2, preferably n² is 1;
R³ is H or —CH₃, preferably R³ is —CH₃;

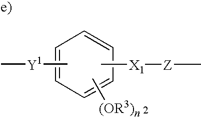

wherein:
Y¹ is —CH₂—CH₂—(CH₂)ₙ^{2a} or —CH═CH—(CH₂)ₙ^{2a} wherein n^{2a} is from 0 to 2, preferably n^{2a} is 0 or 1;
Z, n¹, n², R³ and X₁ are as defined above;

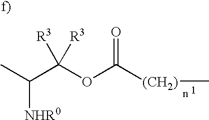

wherein:
n¹ and R³ are as defined above,

### R⁰ is H or —COCH₃;

with the proviso that when Y is selected from the bivalent radicals mentioned under b)-f), then the terminal —ONO₂ group is bound to —(CH₂)ₙ¹;

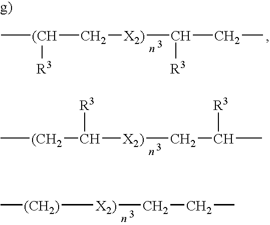

wherein X₂ is —O— or —S—, n³ is an integer from 1 to 6, preferably n³ is 1;
R³ is H or —CH₃, preferably R³ is H;

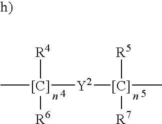

wherein:
n⁴ is an integer from 0 to 10, preferably n⁴ is an integer from 0 to 5;
n⁵ is an integer from 1 to 10, preferably n⁵ is an integer from 1 to 5;
R⁴, R⁵, R⁶, R⁷ are the same or different and are H or straight or branched C₁-C₄ alkyl, preferably R⁴, R⁵, R⁶, K R⁷ are H;
wherein the —ONO₂ group is linked to

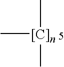

wherein n⁵ is as defined above;
Y² is an heterocyclic saturated, unsaturated or aromatic 5 or 6 members ring, containing one or more heteroatoms selected from nitrogen, oxygen, sulfur,
and is selected from the group consisting of:

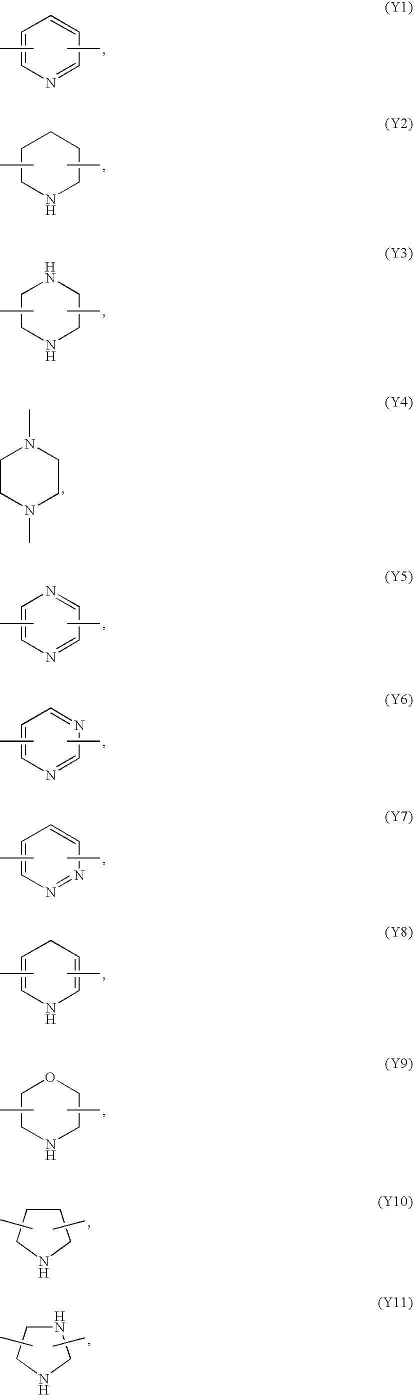

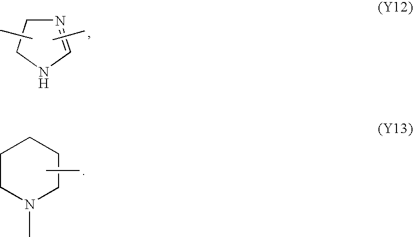

preferably Y² is selected from (Y1), (Y2), (Y4), (Y5), (Y6) or (Y13).

The term “C₁-C₂₀ alkylene” as used herein refers to branched or straight chain C₁-C₂₀ hydrocarbon, preferably having from 1 to 10 carbon atoms such as methylene, ethylene, propylene, isopropylene, n-butylene, pentylene, n-hexylene and the like.

The term “C₁-C₁₀ alkyl” as used herein refers to branched or straight chain alkyl groups comprising one to ten carbon atoms, including methyl, ethyl, n-propyl, isopropyl, n-butyl, isobutyl, t-butyl, pentyl, hexyl, octyl and the like.

The term “cycloalkylene” as used herein refers to ring having from 5 to 7 carbon atoms including, but not limited to, cyclopentylene, cyclohexylene optionally substituted with side chains such as straight or branched (C₁-C₁₀)-alkyl, preferably CH₃.

The term “heterocyclic” as used herein refers to saturated, unsaturated or aromatic 5 or 6 members ring, containing one or more heteroatoms selected from nitrogen, oxygen, sulphur, such as for example pyridine, pyrazine, pyrimidine, pyrrolidine, morpholine, imidazole and the like.

Preferred compounds of formula (I) are those wherein m, R and X are as above defined and Y is a bivalent radical having the following meaning:

a)
straight or branched C₁-C₁₀ alkylene;
straight or branched C₁-C₁₀ alkylene substituted with one or more —ONO₂;

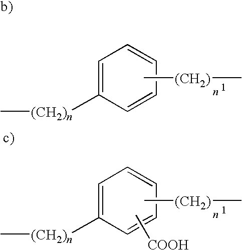

wherein n is an integer from 0 to 5, and n¹ is an integer from 1 to 10;

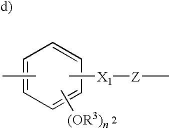

wherein:

### X₁=—OCO—;

Z is —(CH₂)ₙ¹—, n¹ is an integer from 1 to 10;
n² is 1 and R³ is CH₃;

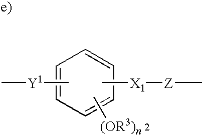

wherein:
Y¹ is —CH₂—CH₂—(CH₂)ₙ^{2a} or —CH═CH—(CH₂)ₙ^{2a} wherein n^{2a} is from 0 to 2, preferably n^{2a} is 0 or 1;

### X₁ is —OCO—;

Z is —(CH₂)ₙ¹—, n¹ is an integer from 1 to 10;
n² is 1, R³ is CH₃;

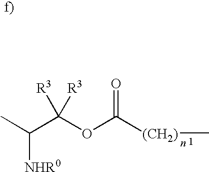

wherein:
n¹ is an integer from 1 to 5, R³ is H and R⁰ is —COCH₃;
with the proviso that when Y is selected from the bivalent radicals mentioned under b)-f), then the terminal —ONO₂ group is bound to —(CH₂)ₙ¹;

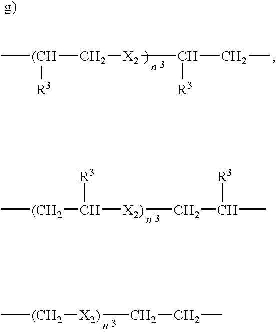

wherein X₂ is —O— or —S—, n³ is 1 and R³ is H.

In another embodiment according to the invention, there is provided a compound and pharmaceutically acceptable salts or stereoisomers thereof according to formula (I):

wherein m is 1 and
in R of formula (II):
A is a carbon atom,

### R¹ is —CH₃,

R′ is an hydrogen atom and
R² is the group —(X—Y—ONO₂) wherein X is —CO— or —COO— and

### Y is:

a)
straight or branched C₁-C₁₀ alkylene,
straight or branched C₁-C₁₀ alkylene substituted with one or more —ONO₂ group;

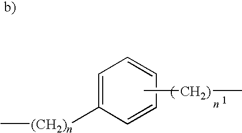

wherein n is an integer from 0 to 5, and n¹ is an integer from 1 to 10;

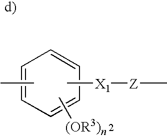

wherein

### X₁=—OCO—;

Z is —(CH₂)ₙ¹— wherein n¹ is an integer from 1 to 10;
n² is 1;

### R³ is —CH₃;

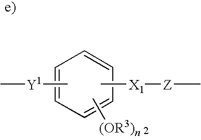

wherein:
Y¹ is —CH₂—CH₂—(CH₂)ₙ^{2a} or —CH═CH—(CH₂)ₙ^{2a} wherein n^{2a} is from 0 to 2, preferably n^{2a} is 0 or 1;

### X₁=—OCO—;

Z is —(CH₂)ₙ¹— wherein n¹ is an integer from 1 to 10;
n² is 1;

### R³ is —CH₃

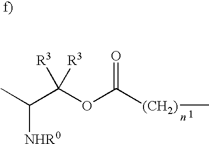

wherein:
n¹ is an integer from 1 to 5, R³ is H and R⁰ is —COCH₃;
with the proviso that when Y is selected from the bivalent radicals mentioned under b)-f), then the terminal —ONO₂ group is bound to —(CH₂)ₙ¹;

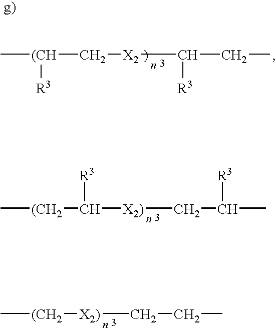

wherein X₂ is —O— or —S—,
n³ is 1;

### R³ is H.

In another embodiment according to the invention, there is provided a compound and pharmaceutically acceptable salts or stereoisomers thereof according to formula (I):

wherein m is 1 and
in R of formula (II):
A is a carbon atom,

### R¹ is —CH₃,

R² is an hydrogen atom and
R′ is the group —(X—Y—ONO₂) wherein X is —CO— or —COO— and

### Y is:

a)
straight or branched C₁-C₁₀ alkylene;
straight or branched C₁-C₁₀ alkylene substituted with one or more —ONO₂ group;

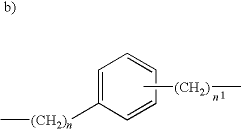

wherein n is an integer from 0 to 5, and n¹ is an integer from 1 to 10;

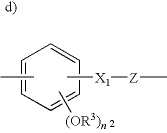

wherein

### X₁ is —OCO—;

Z is —(CH₂)ₙ¹— wherein n¹ is an integer from 1 to 10;
n² is an integer from 0 to 2, preferably n² is 1;
R³ is H or —CH₃, preferably R³ is —CH₃;

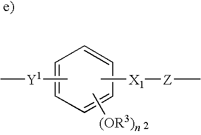

wherein:
Y¹ is —CH₂—CH₂—(CH₂)ₙ^{2a} or —CH═CH—(CH₂)ₙ^{2a} wherein n^{2a} is from 0 to 2, preferably n^{2a} is 0 or 1;

### X₁ is —OCO—;

Z is —(CH₂)ₙ¹— wherein n¹ is an integer from 1 to 20;
n² is 1;

### R³ is —CH₃;

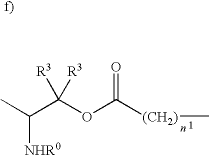

wherein:
n¹ is an integer from 1 to 5, R³ is H and R⁰ is —COCH₃;
with the proviso that when Y is selected from the bivalent radicals mentioned under b)-f), then the terminal —ONO₂ group is bound to —(CH₂)ₙ¹;

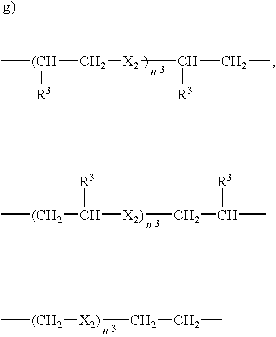

wherein X₂ is —O— or —S—, n³ is 1;
R³ is H or —CH₃, preferably R³ is H;

The following are preferred compounds according to the present invention:

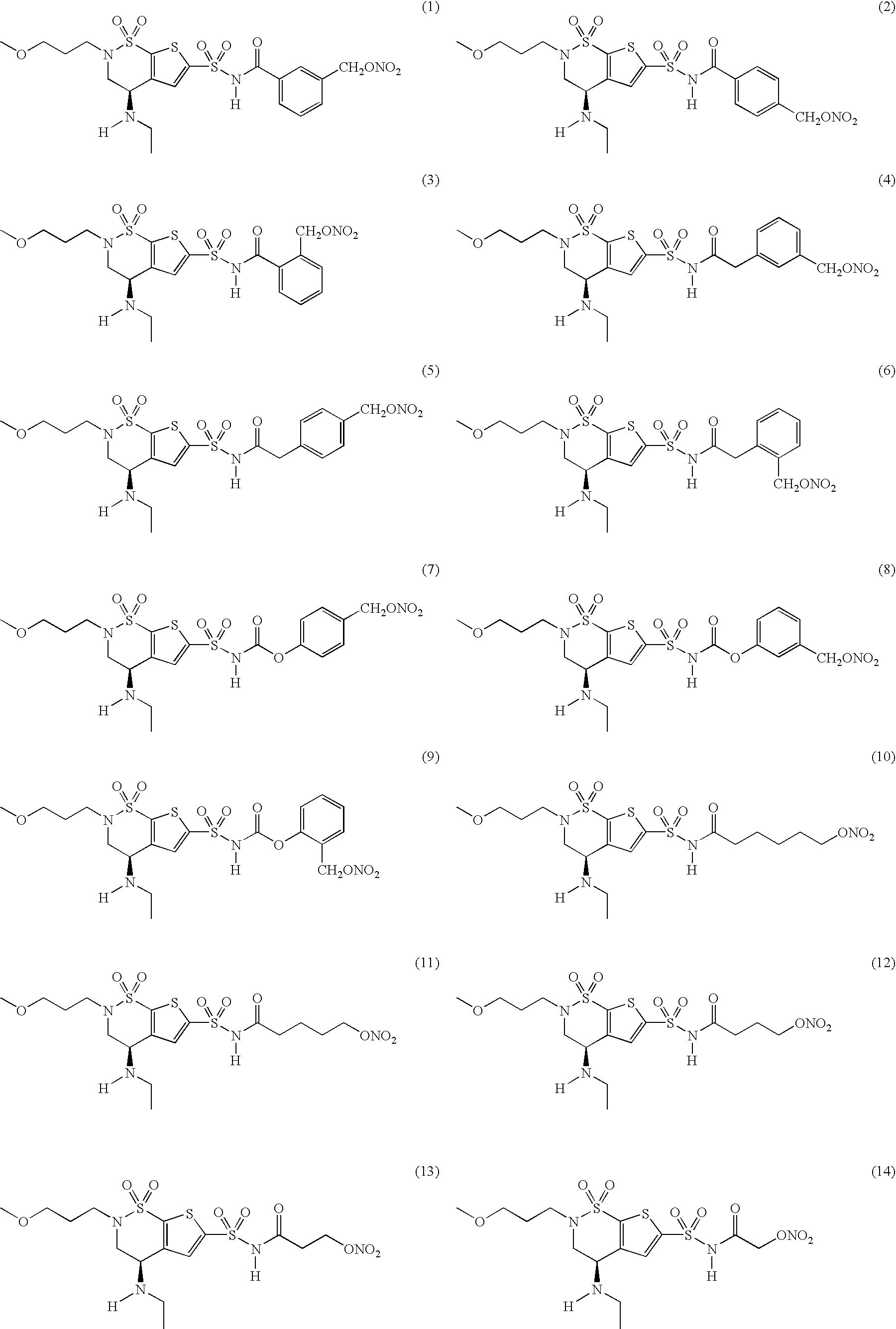

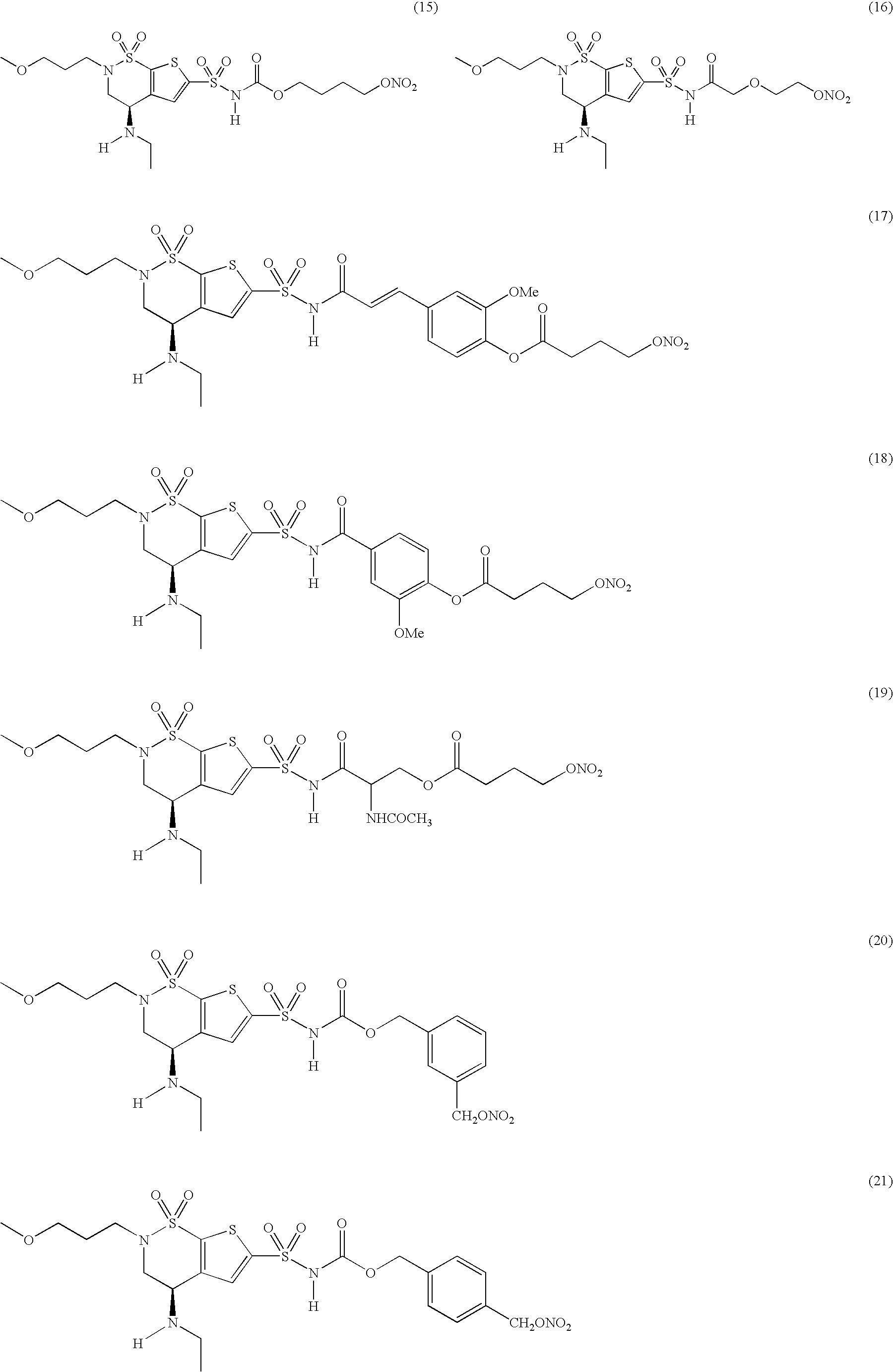

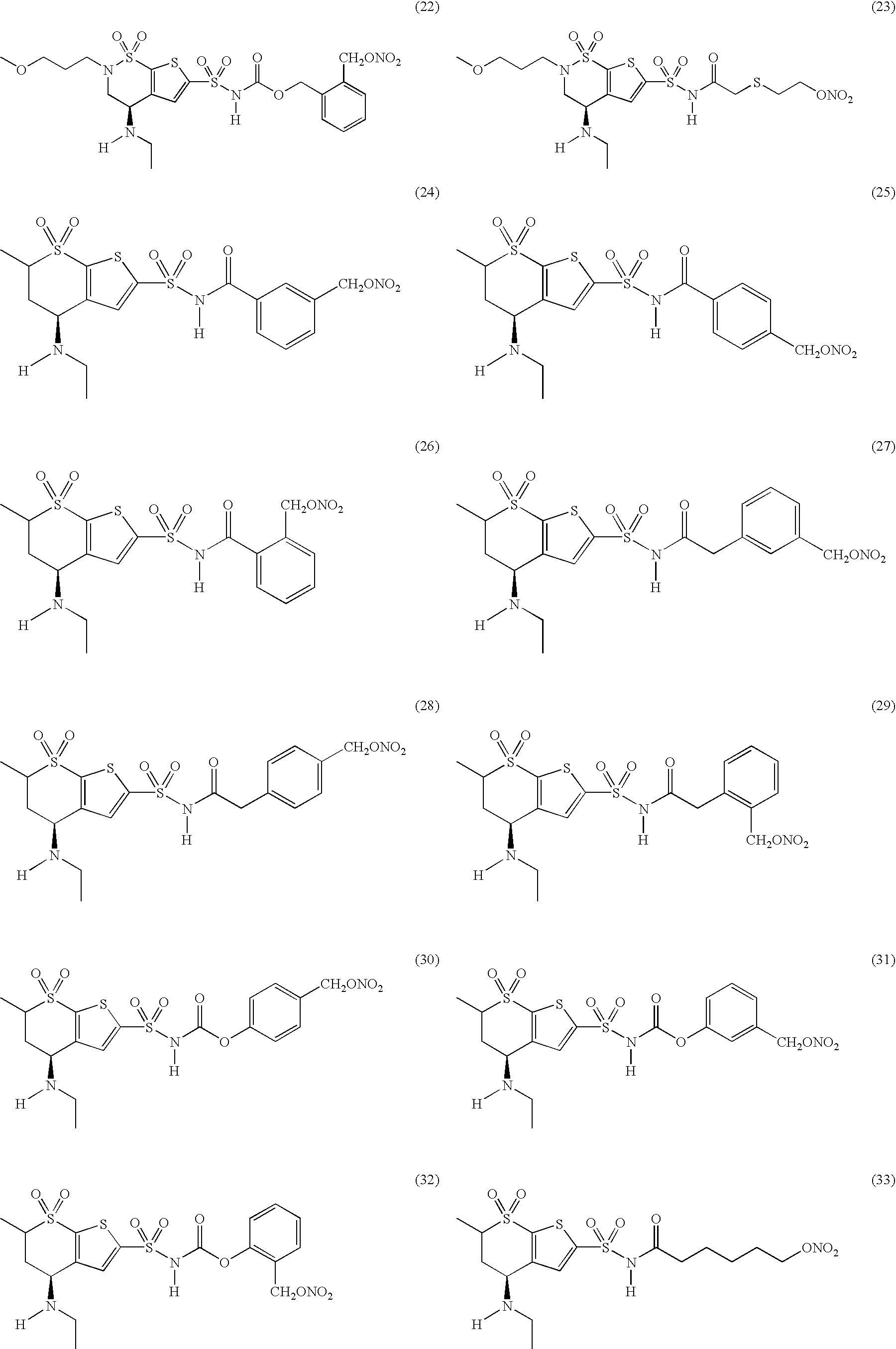

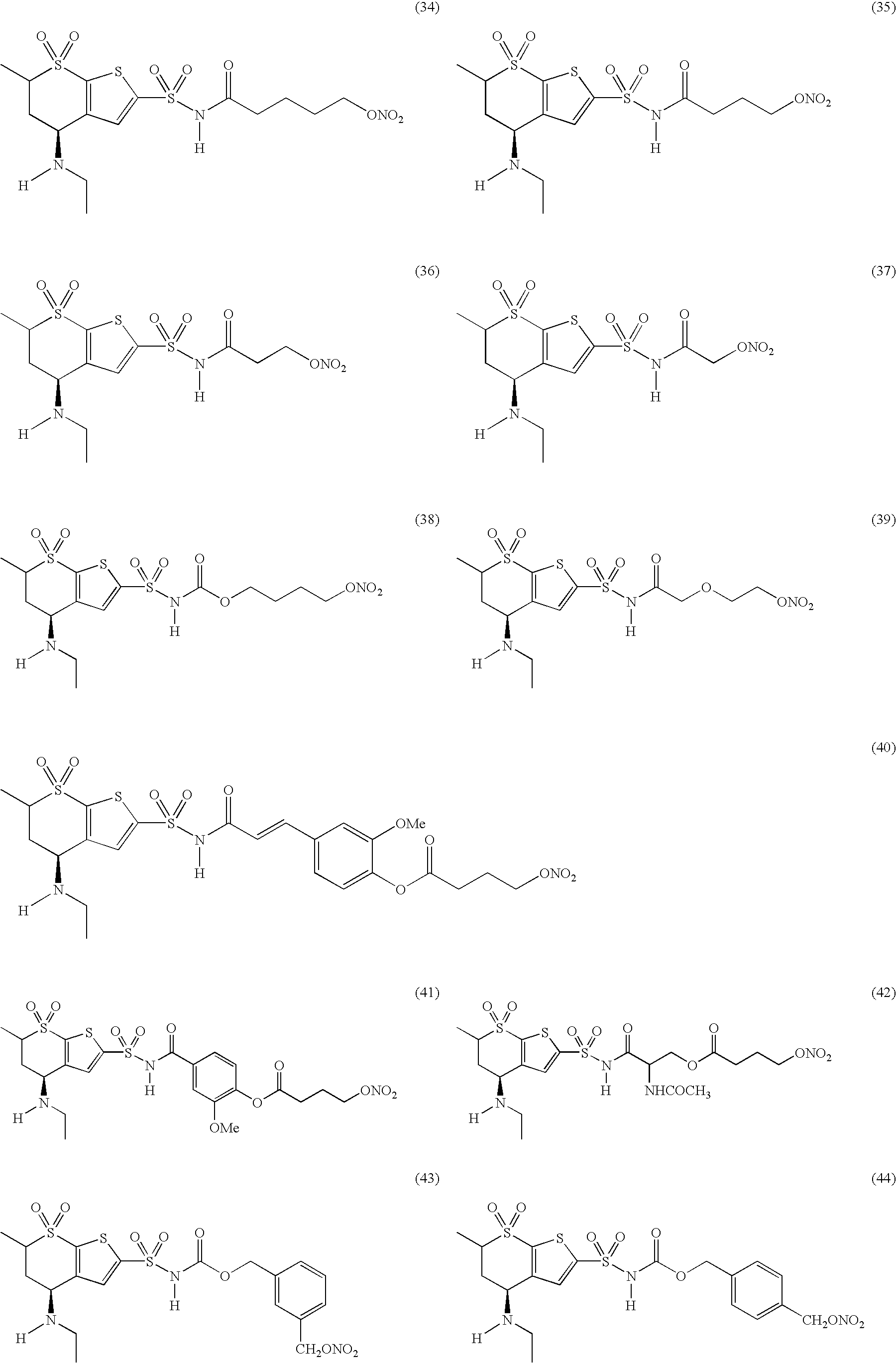

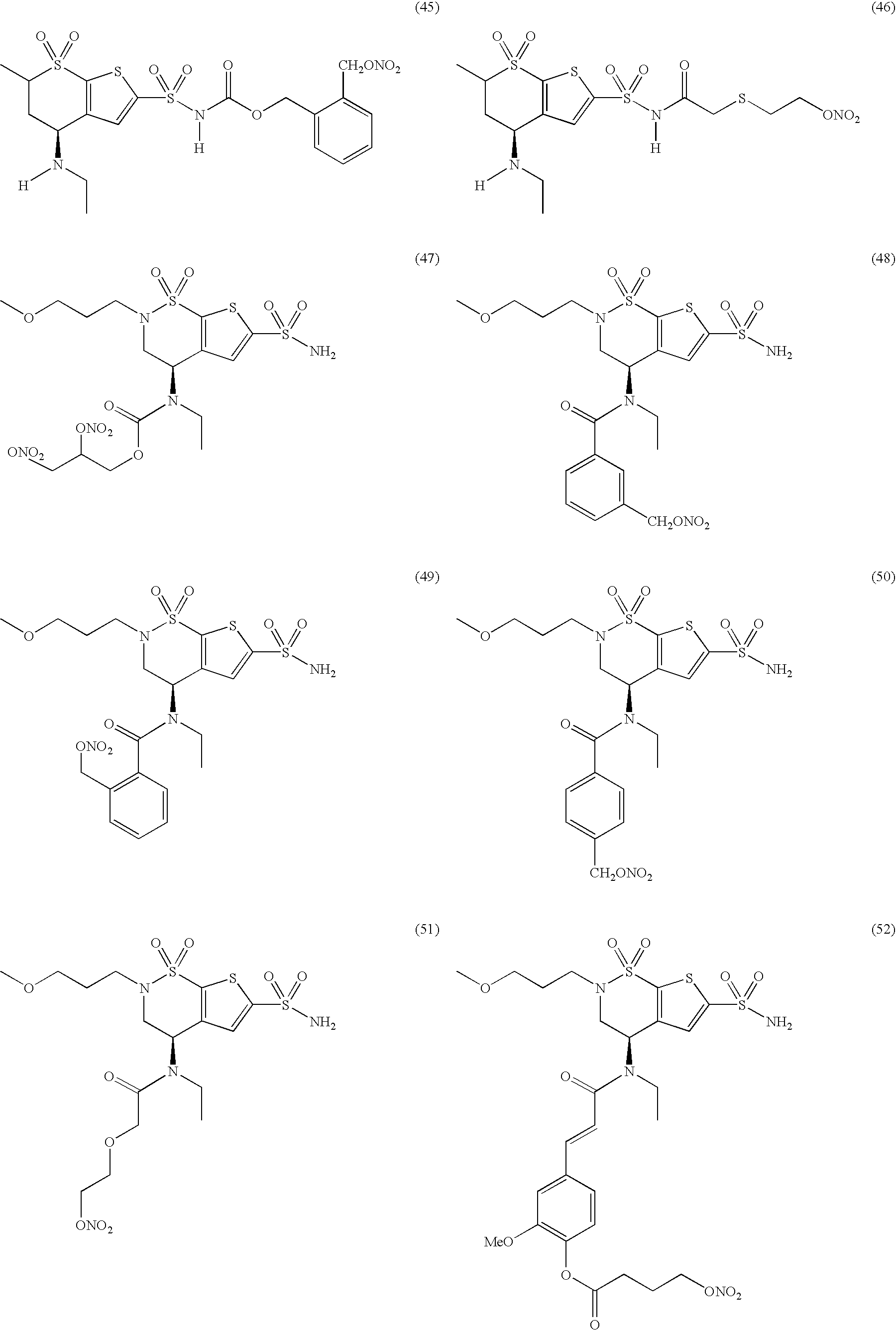

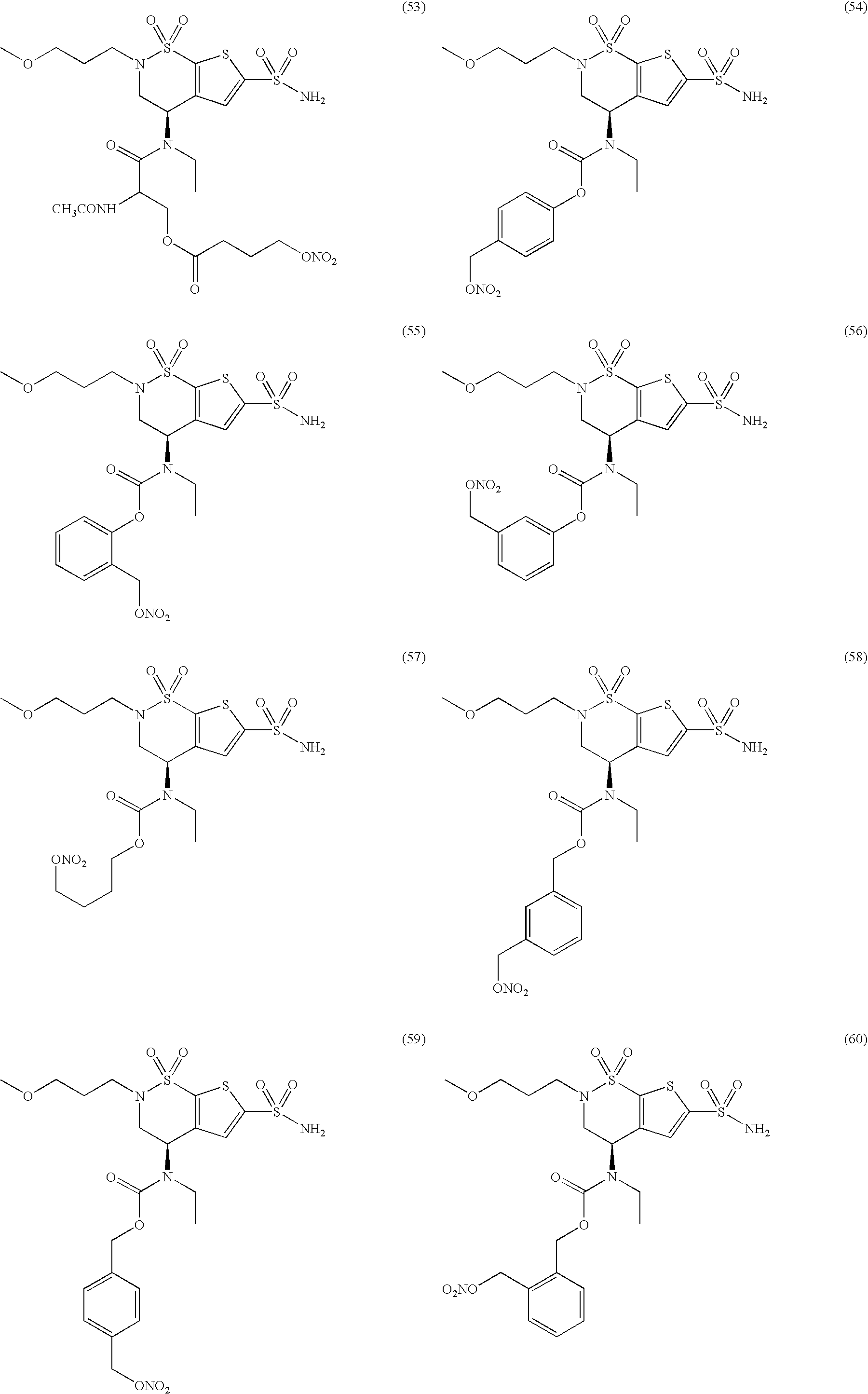

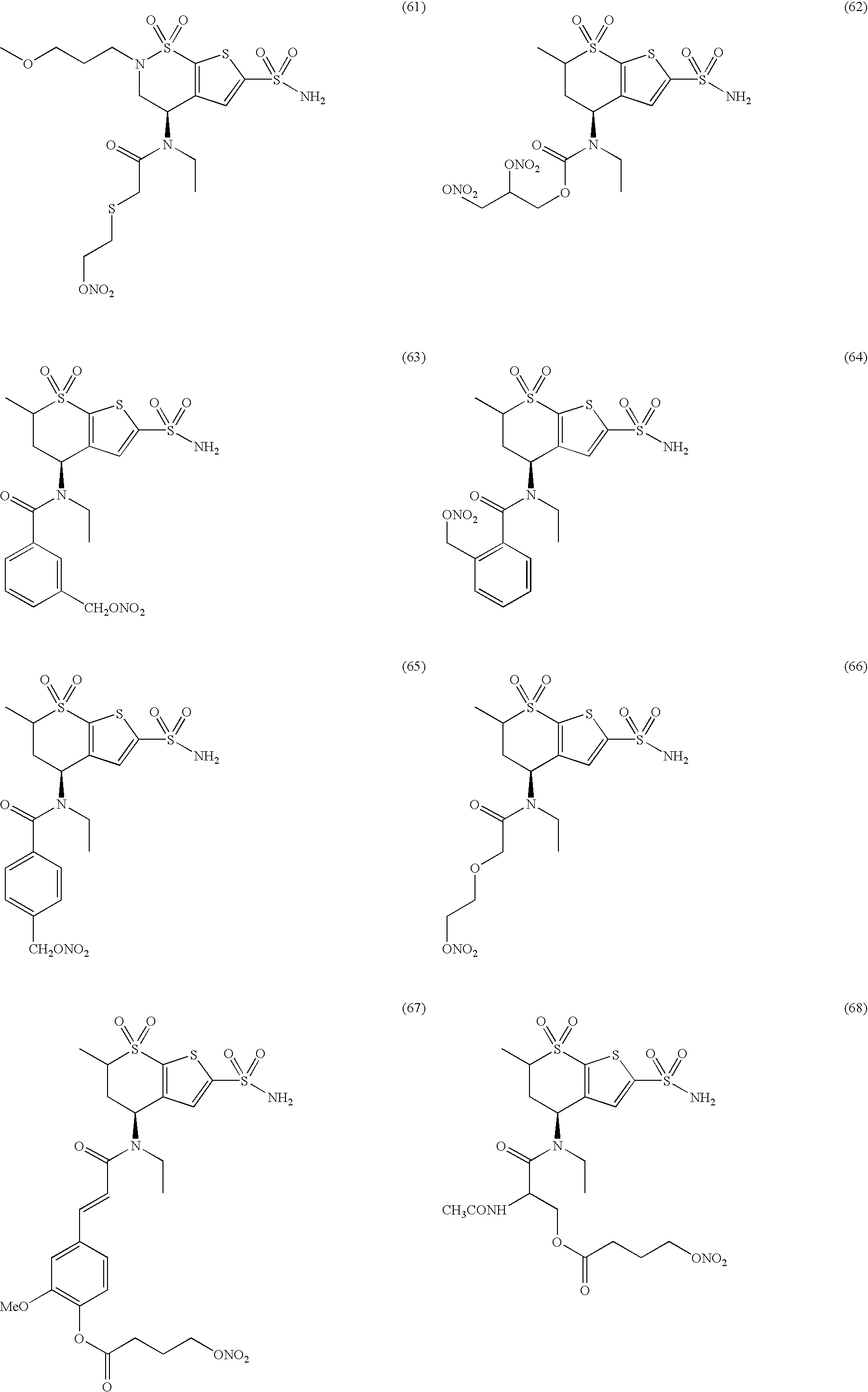

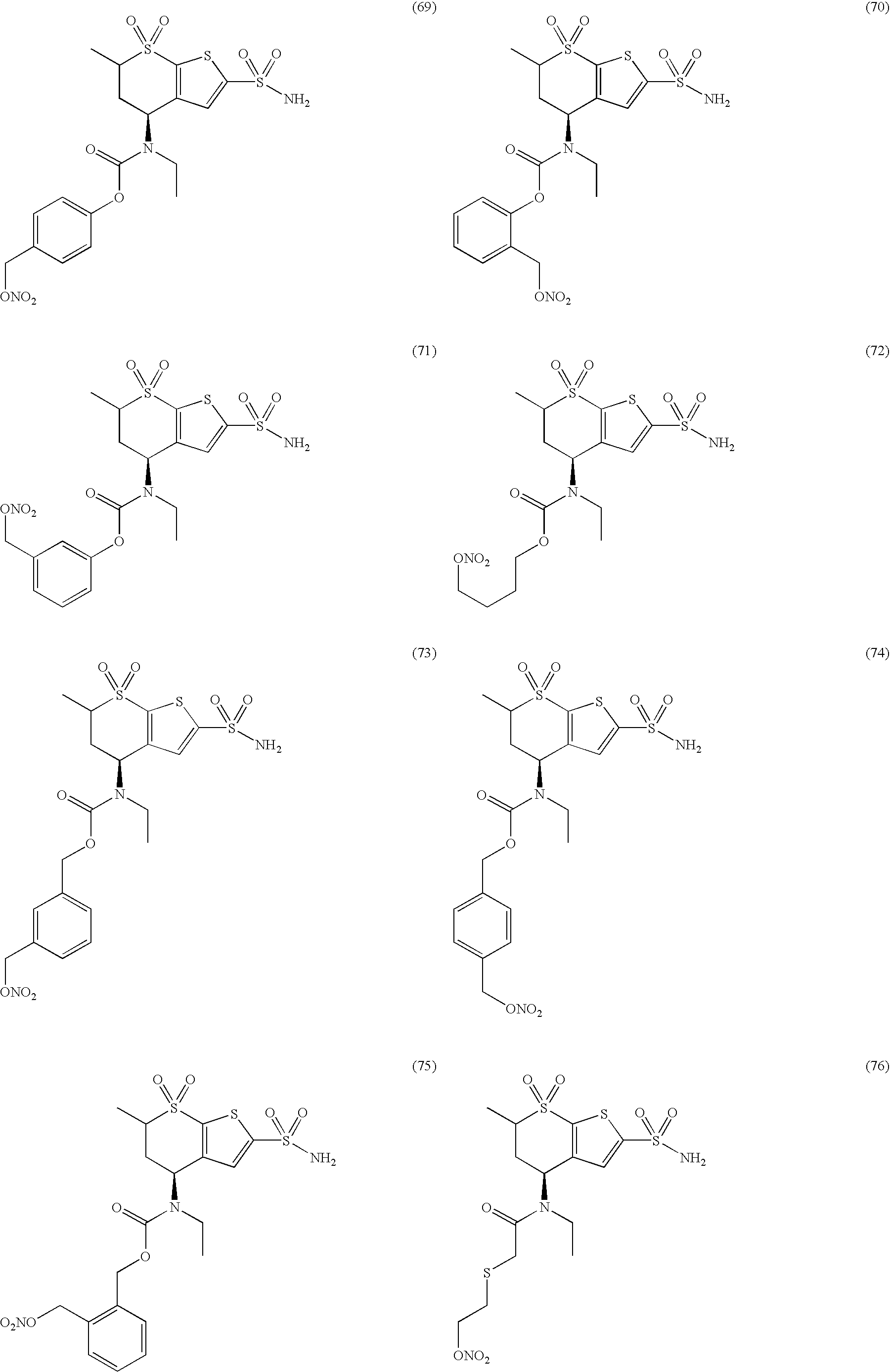

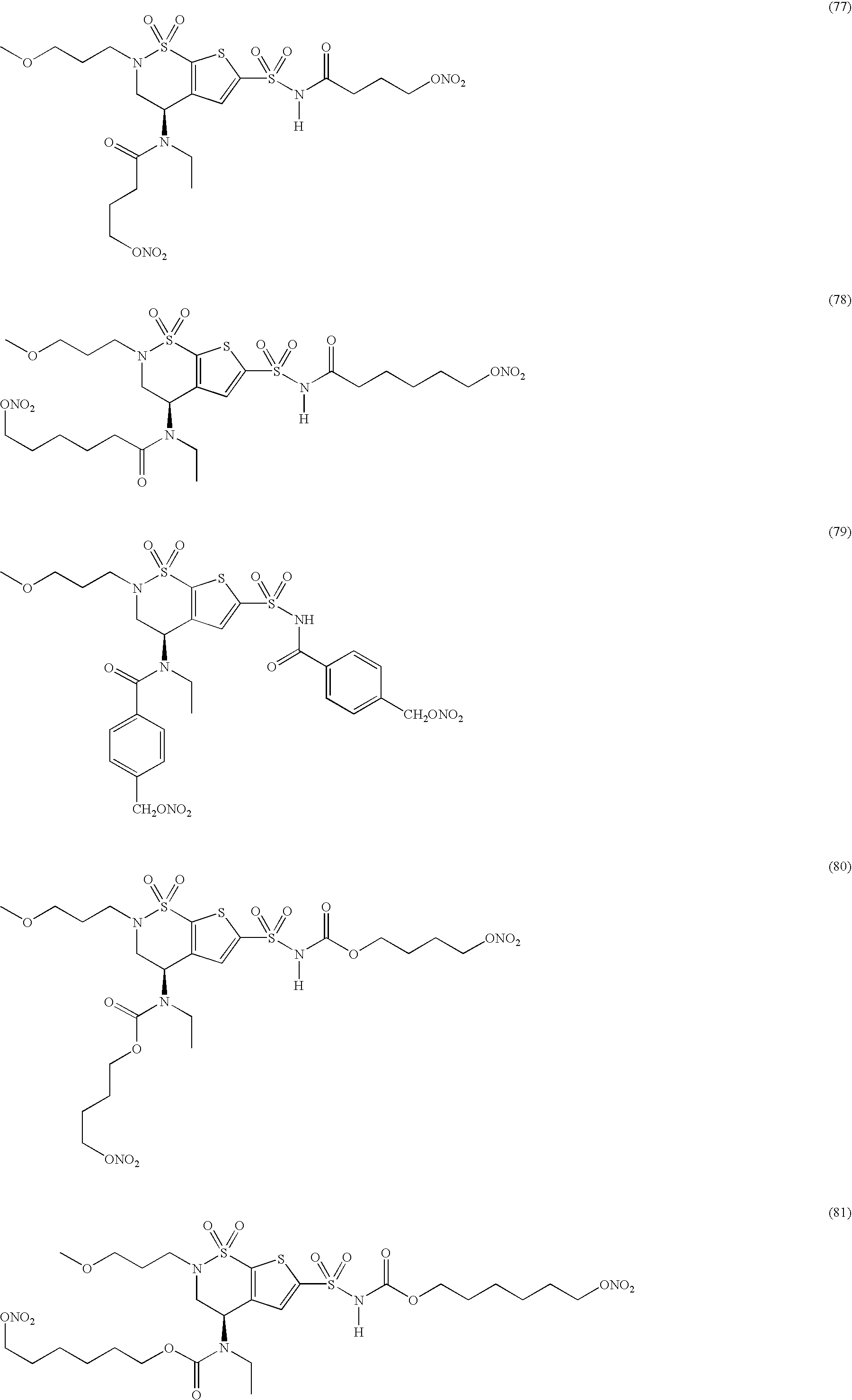

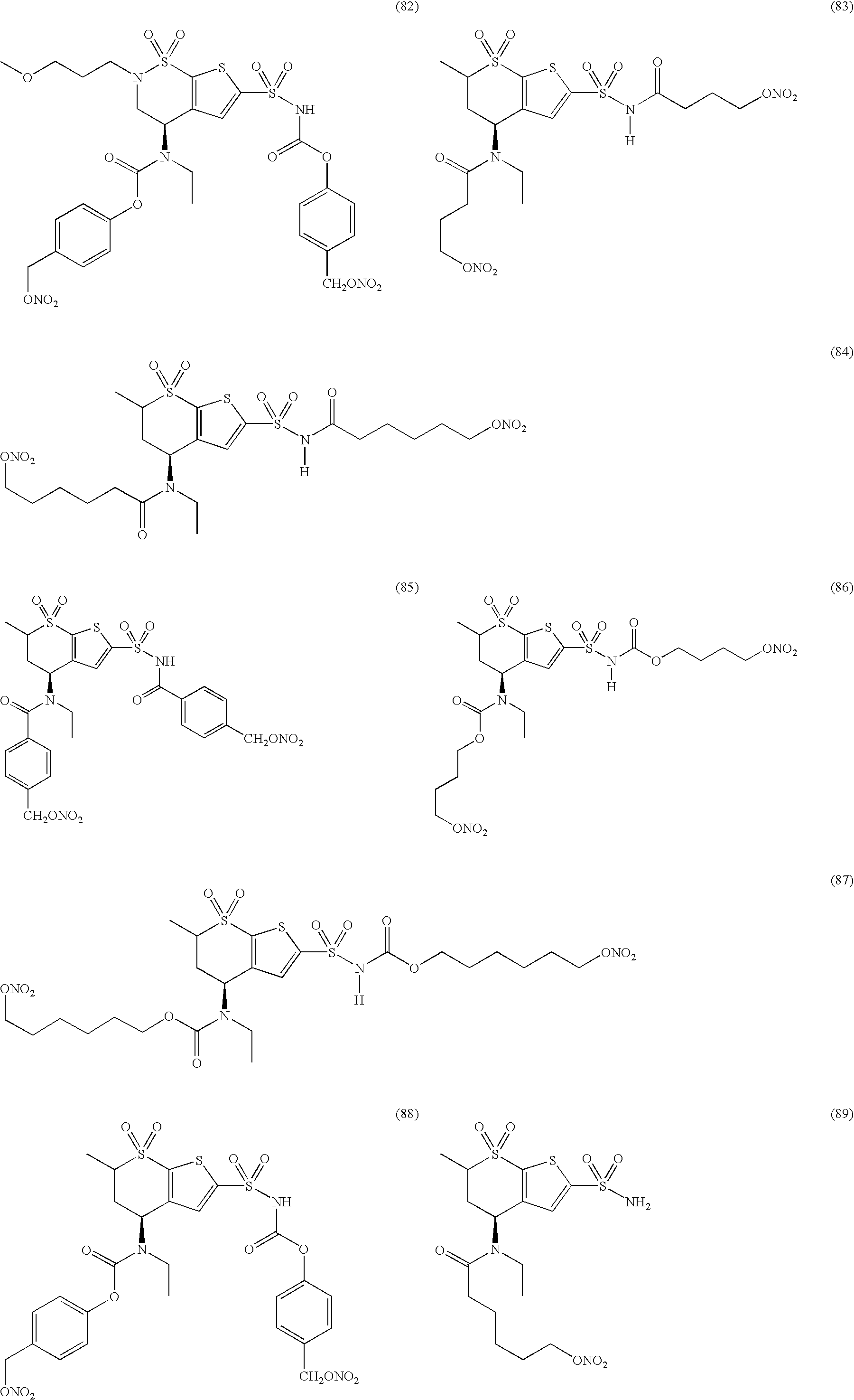

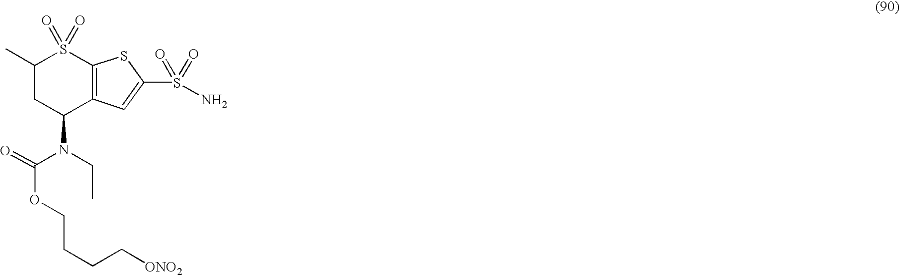

As stated above, the invention includes also the pharmaceutically acceptable salts of the compounds of formula (I) and stereoisomers thereof.

Examples of pharmaceutically acceptable salts are either those with inorganic bases, such as sodium, potassium, calcium and aluminium hydroxides, or with organic bases, such as lysine, arginine, triethylamine, dibenzylamine, piperidine and other acceptable organic amines.

The compounds according to the present invention, when they contain in the molecule one salifiable nitrogen atom, can be transformed into the corresponding salts by reaction in an organic solvent such as acetonitrile, tetrahydrofuran with the corresponding organic or inorganic acids.

Examples of organic acids are: oxalic, tartaric, maleic, succinic, citric acids. Examples of inorganic acids are: nitric, hydrochloric, sulphuric, phosphoric acids. Salts with nitric acid are preferred.

The compounds of the invention which have one or more asymmetric carbon atoms can exist as optically pure enantiomers, pure diastereomers, enantiomers mixtures, diastereomers mixtures, enantiomer racemic mixtures, racemates or racemate mixtures. Within the scope of the invention are also all the possible isomers, stereoisomers and their mixtures of the compounds of formula (I), including mixtures enriched in a particular isomer.

As mentioned above, objects of the present invention are also pharmaceutical compositions containing at least a compound of the present invention of formula (I) together with non toxic adjuvants and/or carriers usually employed in the pharmaceutical field.

The preferred route of administration is topical.

The compounds of the present invention can be administered as solutions, suspensions or emulsions (dispersions) in an ophthalmically acceptable vehicle. The term “ophthalmically acceptable vehicle” as used herein refers to any substance or combination of substances which are non-reactive with the compounds and suitable for administration to patient.

Preferred are aqueous vehicles suitable for topical application to the patient's eyes.

Other ingredients which may be desirable to use in the ophthalmic compositions of the present invention include antimicrobials, preservatives, co-solvents, surfactants and viscosity building agents.

The invention also relates to a method for treating glaucoma or ocular hypertension, said method consisting in contacting an effective intraocular pressure reducing amount of a composition with the eye in order to reduce eye pressure and to maintain said pressure on a reduced level.

The invention also relates to the use of the compounds of formula (I) as antiglaucoma agents or as drugs able to reduce the intraocular pressure and to maintain said pressure on a reduced level.

The invention also relates to the use of the compounds of formula (I) as drugs for treating glaucoma, ocular hypertension, age-related macular degeneration, diabetic macular edema, diabetic retinopathy, hypertensive retinopathy and retinal vasculopathies.

The doses of carbonic anydrase inhibitors nitroderivatives can be determined by standard clinical techniques and are in the same range or less than those described for the corresponding underivatized, commercially available, dorzolamide and brinzolamide as reported in the: Physician's Desk Reference, Medical Economics Company, Inc., Oradell, N.J., 58^{th} Ed., 2004; The pharmacological basis of therapeutics, Goodman and Gilman, J. G. Hardman, L. e. Limbird, Tenth Ed.

It is further contemplated that the compounds of the present invention can be used with other medicaments known to be useful in the treatment of glaucoma or ocular hypertension, either separately or in combination. For example the compounds of the present invention can be combined with (i) beta-blockers, such as timolol, betaxolol, levobunolol and the like (see U.S. Pat. No. 4,952,581); (ii) prostaglandin analogs, such as bimatoprost, latanoprost, travoprost or unoprostone (iii) α-adrenergic agonists including clonidine derivatives, such as apraclonidine or brimonidine (see U.S. Pat. No. 5,811,443). Also contemplated is the combination with nitrooxy derivatives of the above reported compounds, for example nitrooxy derivatives of beta-blockers which are disclosed in U.S. Pat. No. 6,242,432, or nitrooxy derivatives of prostaglandin analogs disclosed in WO 2005/068421.

### Synthesis Procedure

1. The compound of general formula (I) as above defined wherein:

X is —CO—, m is 1, R and Y are as above defined, wherein R′ is H and R² is a free valence can be obtained by a process comprising:

1a. reacting a compound of formula B with a compound of formula (IIIa):

B+HOOC—Y—ONO₂ (IIIa)

wherein Y is as above defined; B is equal to R with R² being H and R′ is PG wherein PG is a sulfonamido protecting group such as dimethylformamidine, in presence of a condensing agent like dicyclohexylcarbodiimide (DCC), N-(3-dimethylaminopropyl)-N′-ethylcarbodiimide hydrochloride (EDAC) or N,N′-carbonyldiimidazole (CDI) or other known condensing reagents such as HATU in solvent such as DMF, THF, chloroform at a temperature in the range from −5° C. to 50° C. in the presence or not of a base as for example DMAP and deprotecting the compound by reaction with hydrochloric acid in methanol.

The nitric acid ester compounds of formula (IIIa) can be obtained from the corresponding alcohols of formula HOOC—Y—OH (IIIb), that are commercially available, by reaction with nitric acid and acetic anhydride in a temperature range from −50° C. to 0° C. or reacting the corresponding halogen derivatives of formula HOOC—Y-Hal (IIIc) wherein Hal is an alogen atom preferable Cl, Br, I, that are commercially available, with AgNO₃ as described in WO 2006/008196.

Compounds of formula B wherein R′ and R² are H, are known as dorzolamide and brinzolamide

1b. reacting a compound of formula B as above defined with a compound of formula (IIId):

Hal-CO—Y—ONO₂ (IIId)

wherein Y is as above defined; Hal is an Halogen atom. The reaction is generally carried out in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C. and deprotecting the compound by reaction with hydrochloric acid in methanol.

The compounds of formula (IIId) can be obtained from the corresponding compound (IIIa) by well known reactions, for example by reaction with thionyl or oxalyl chloride, halides of P^{III} or P^{V} in solvents inert such as toluene, chloroform, DMF, etc.

1c. reacting a compound of formula R—X—Y-Hal (IVa), wherein R, X, Y and Hal are as defined in 1-1a., with AgNO₃ and deprotecting the compound by reaction with hydrochloric acid in methanol. Compounds (IVa) can be obtained by reacting compound B with compounds (IIIc), as above defined, with a condensing reagent such as DCC or CDI as above described.

1d. reacting a compound of formula R—X—Y—OH (Va), wherein R, X and Y are as defined in 1., with triflic anhydride/tetraalkylammonium nitrate salt in an aprotic polar/non-polar solvent such as DMF, THF or CH₂Cl₂ at temperatures range between −60° to 65° C. and deprotecting the compound by reaction with hydrochloric acid in methanol. Compounds (Va) can be obtained by reacting compound B with compounds (IIIb), as above defined, with a condensing reagent as above described.

2. The compound of general formula (I) as above defined wherein:

X is —CO—, m is 1, R is as above defined, wherein R′ is H and R² is a free valence and Y is a straight or branched C₁-C₂₀ alkyl substituted by a —ONO₂ group can be obtained by a process comprising:

2a. reacting a compound of formula R—X—Y′ (Via) wherein Y′ is straight or branched C₁-C₂₀ alkenyl, R is as above defined, wherein R² is H and R′ is PG wherein PG is a sulfonamido protecting group such as dimethylformamidine, with iodine and silver nitrate in acetonitrile at a temperature between −20° C. and 80° C. and deprotecting the compound by reaction with hydrochloric acid in methanol.

Compound (VIa) can be obtained by reacting compound B with compound HOOC—Y′ (VIIa) in presence of a condensing agent like dicyclohexylcarbodiimide (DCC), N-(3-dimethylaminopropyl)-N′-ethylcarbodiimide hydrochloride (EDAC) or N,N′-carbonyldiimidazole (CDI) or other known condensing reagents such as HATU in solvent such as DMF, THF, chloroform at a temperature in the range from −5° C. to 50° C. in the presence or not of a base as for example DMAP.

Compound (VIIa) are commercially available.

3. The compound of general formula (I) as above defined wherein:

X is —COO—; m is 1, R and Y are as above defined, wherein R′ is H and R² is a free valence can be obtained by a process comprising:

3a. reacting a compound of formula B with a compound of formula (VIIIa):

B+Hal-X—Y—ONO₂ (VIIIa)

wherein B is equal to R with R² being H and R′ being PG wherein PG is a sulfonamido protecting group such as dimethylformamidine; X is —COO—; Hal and Y are as above described. The reaction is generally carried out in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C. and deprotecting the compound by reaction with hydrochloric acid in methanol.

The compounds of formula (VIIIa) can be obtained from the corresponding alcohols HO—Y—ONO₂ (VIIIb) by reaction with triphosgene in presence of an organic base. The nitric acid ester compounds of formula (VIIIb) can be obtained from the corresponding alcohols of formula HO—Y—OH (VIIIc), that are commercially available, by reaction with nitric acid and acetic anhydride in a temperature range from −50° C. to 0° C. or reacting the corresponding halogen derivatives of formula HO—Y-Hal (VIIId) wherein Hal is an halogen atom preferable Cl, Br, I, that are commercially available, with AgNO₃ as already described in the international application No. WO 2006/008196.

3b. reacting a compound of formula R—X—Y-Hal (IXa) wherein R, X, Y and Hal are as above defined, with AgNO₃ and deprotecting the compound by reaction with hydrochloric acid in methanol.

The compounds of formula (IXa) can be obtained by reacting compound B with compounds Hal-X—Y-Hal (Xa). The reaction is generally carried out in presence of an inorganic or organic base in an aprotic polar/non-polar solvent such as DMF, THF or CH₂Cl₂ at temperatures range between 0°-65° C. as above described.

Compound (Xa) are commercially available.

4. The compound of general formula (I) as above defined wherein:

X is —COO—, m is 1, R is as above defined, R′ is H and R² is a free valence and Y is a straight or branched C₁-C₂₀ alkyl substituted by a —ONO₂ group can be obtained by a process comprising:

4a. reacting a compound of formula R—X—Y′ (XIa) wherein Y′ is straight or branched C₁-C₂₀ alkenyl, R is as above defined, R² is H and R′ is PG wherein PG is a sulfonamido protecting group such as dimethylformamidine, with iodine and silver nitrate in acetonitrile at a temperature between −20° C. and 80° C. and deprotecting the compound by reaction with hydrochloric acid in methanol.

Compound (XIa) can be obtained by reacting compound B with compound Hal-X—Y′ (XIIa) in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C.

Compound (XIIa) are commercially available.

5. The compound of general formula (I) as above defined wherein:

X is —CO—, m is 1, R and Y are as above defined, wherein R² is H and R′ is a free valence can be obtained by a process comprising:

5a. reacting a compound of formula B with a compound of formula (IIIa):

HOOC—Y—ONO₂ (IIIa)

wherein Y is as above defined; B is equal to R with R² being PG₁ wherein PG₁ is an amino protecting group such as Fmoc or Alloc and R′ is H, in presence of a condensing agent like dicyclohexylcarbodiimide (DCC), N-(3-dimethylaminopropyl)-N′-ethylcarbodiimide hydrochloride (EDAC) or N,N′-carbonyldiimidazole (CDI) or other known condensing reagents such as HATU in solvent such as DMF, THF, chloroform at a temperature in the range from −5° C. to 50° C. in the presence or not of a base as for example DMAP and deprotecting the compound by reaction with a organic base such as piperidine in a solvent as acetonitrile at temperature range between 20°-40° C., or by reaction with morfoline in the of presence of palladium tetrakis in tetrahydrofurane at temperature range between 20°-40° C.

The compound B as above defined can be obtained from the compound B as defined in 1.1a. protecting the amino with a group PG₁ wherein PG₁ is as above described and deprotecting the sulphonamide group by reaction with hydrochloric acid in methanol.

5b. reacting a compound of formula B as above defined with a compound of formula (IIId):

B+Act-CO—Y—ONO₂ (XIIId)

wherein Y is as above defined; Act is an Halogen atom or a carboxylic acid activating group used in peptide chemistry as:

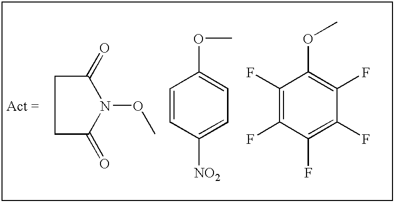

The reaction is generally carried out in presence of a inorganic or organic base in an aprotic polar/non-polar

solvent such as DMF, THF or CH₂Cl₂ at temperatures range between 0°-65° C. or in a double phase system H₂O/Et₂O at temperatures range between 20°-40° C.; or in the presence of DMAP and a Lewis acid such as Sc(OTf)₃ or Bi(OTf)₃ in solvents such as DMF, CH₂Cl₂ and deprotecting the compound by reaction with a organic base such as piperidine in a solvent as acetonitrile at temperature range between 20°-40° C., or by reaction with morfoline in the of presence of palladium tetrakis in tetrahydrofurane at temperature range between 20°-40° C.

The compounds of formula (XIIId) can be obtained as described in WO 2006/008196.

5c. reacting a compound of formula R—X—Y-Hal (XIVa), wherein R, X, Y and Hal are as defined in 5-1a., with AgNO₃ and deprotecting the compound by reaction with a organic base such as piperidine in a solvent as acetonitrile at temperature range between 20°-40° C., or by reaction with morfoline in the of presence of palladium tetrakis in tetrahydrofurane at temperature range between 20°-40° C.

Compounds (XIVa) can be obtained by reacting compound B with compounds (IIIc), as above defined, with a condensing reagent such as DCC or CDI as above described.

5d. reacting a compound of formula R—X—Y—OH(XVa), wherein R, X and Y are as defined in 5, with triflic anhydride/tetraalkylammonium nitrate salt in an aprotic polar/non-polar solvent such as DMF, THF or CH₂Cl₂ at temperatures range between −60° to 65° C. and deprotecting the compound by reaction with a organic base such as piperidine in a solvent as acetonitrile at temperature range between 20°-40° C., or by reaction with morfoline in the of presence of palladium tetrakis in tetrahydrofurane at temperature range between 20°-40° C.

Compounds (XVa) can be obtained by reacting compound B with compounds (IIIb), as above defined, with a condensing reagent as above described.

6. The compound of general formula (I) as above defined wherein:

X is —CO—, m is 1, R is as above defined, R² is H and R′ is a free valence and Y is a straight or branched C₁-C₂₀ alkyl substituted by a —ONO₂ group can be obtained by a process comprising:

6a. reacting a compound of formula R—X—Y′ (XVIa) wherein Y′ is straight or branched C₁-C₂₀ alkenyl, R is as above defined, R′ is H and R² is PG₁ wherein PG₁ is an amino protecting group such as Fmoc or Alloc and R′ is H, with iodine and silver nitrate in acetonitrile at a temperature between −20° C. and 80° C. and deprotecting the compound by reaction with a organic base such as piperidine in a solvent as acetonitrile at temperature range between 20°-40° C., or by reaction with morfoline in the of presence of palladium tetrakis in tetrahydrofurane at temperature range between 20°-40° C.

Compound (XVIa) can be obtained by reacting compound B with compound HOOC—Y′ (XVIIa) in presence of a condensing agent like dicyclohexylcarbodiimide (DCC), N-(3-dimethylaminopropyl)-N′-ethylcarbodiimide hydrochloride (EDAC) or N,N′-carbonyldiimidazole (CDI) or other known condensing reagents such as HATU in solvent such as DMF, THF, chloroform at a temperature in the range from −5° C. to 50° C. in the presence or not of a base as for example DMAP.

Compound (XVIIa) are commercially available.

7. The compound of general formula (I) as above defined wherein:

X is —COO—; m is 1, R and Y are as above defined, R² is H and R′ is a free valence can be obtained by a process comprising:

7a. reacting a compound of formula B with a compound of formula (VIIIa):

B+Hal-X—Y—ONO₂ (VIIIa)

wherein B is equal to R with R² being PG₁ wherein PG₁ is an amino protecting group such as Fmoc or Alloc and R′ is H; X is —COO—; Hal and Y are as above described.

The reaction is generally carried out in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C. and deprotecting the compound by reaction with a organic base such as piperidine in a solvent as acetonitrile at temperature range between 20°-40° C., or by reaction with morfoline in the of presence of palladium tetrakis in tetrahydrofurane at temperature range between 20°-40° C.

7b. reacting a compound of formula R—X—Y-Hal (XIXa) wherein R, X, Y and Hal are as above defined, with AgNO₃ and deprotecting the compound as above described.

The compounds of formula (XIXa) can be obtained by reacting compound B with compounds Hal-X—Y-Hal (Xa). The reaction is generally carried out in presence of an inorganic or organic base in an aprotic polar/non-polar solvent such as DMF, THF or CH₂Cl₂ at temperatures range between 0°-65° C. as above described.

8. The compound of general formula (I) as above defined wherein:

X is —COO—, m is 1, R is as above defined, R² is H and R′ is a free valence and Y is a straight or branched C₁-C₂₀ alkyl substituted by a —ONO₂ group can be obtained by a process comprising:

8a. reacting a compound of formula R—X—Y′ (XXa) wherein Y′ is straight or branched C₁-C₂₀ alkenyl, R is as above defined, R′ is H and R² is PG wherein PG is a sulfonamido protecting group such as dimethylformamidine, with iodine and silver nitrate in acetonitrile at a temperature between −20° C. and 80° C. and deprotecting the compound by reaction with hydrochloric acid in methanol.

Compound (XXa) can be obtained by reacting compound B with compound Hal-X—Y′ (XXIa) in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C.

Compound (XXIa) are commercially available.

9. The compound of general formula (I) as above defined wherein:

X is —CO— or —COO—, m is 2, R and Y are as above defined, wherein R′ and R² are a free valence can be obtained by a process as above described in 1-8.

### EXAMPLE 1

### Synthesis of Compound of Formula (33)

### (4S-trans)-4-(ethylamino)-5,6-dihydro-6-methyl-4H-thieno[2,3-b]thiopyran-2-sulfonylamido-N((6-nitrooxy)hexanoyl)-7,7-dioxide

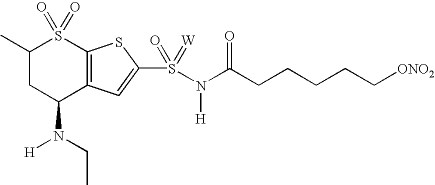

### A) (4S-trans)-4-(ethylamino)-5,6-dihydro-6-methyl-4H-thieno[2,3-b]thiopyran-2-sulfonylamido-N-(dimethylamino methylen)-7,7-dioxide

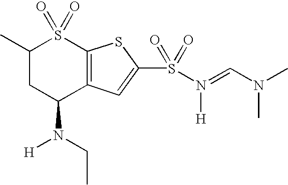

To a solution of dorzolamide hydrochloride (722 mg, 2.0 mmol) in DMF (1.4 mL), triethyl amine (0.31 mL, 2.2 mmol) and N,N-dimethylformamide dimethylacetal (0.32 mL, 2.4 mmol) were added. The reaction was monitored by TLC eluting with CH₂Cl₂/MeOH 95/5 (Rf compound A=0.41). After stirring for 3 h at rt under nitrogen, the reaction was cooled to 0° C. using an iced water bath and water (10 mL) was added. The aqueous solution was extracted with ethyl acetate (3×10 mL). The combined AcOEt extracts were washed with water (10 mL), dried over Na₂SO₄, and concentrated in vacuo to give the imine derivative A (608 mg, 80%) as a white solid.

### B) (4S-trans)-4-(amino-N-ethyl-N-Fmoc)-5,6-dihydro-6-methyl-4H-thieno[2,3-b]thiopyran-2-sulfonylamido-N-(dimethyl aminomethylen)-7,7-dioxide

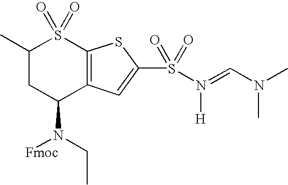

To a solution of compound A (2.65 g, 7 mmol) in dioxane (18 mL) and a 10% aqueous solution of Na₂CO₃ (18 mL), cooled to 0° C., a solution of FmocCl (1.8 g, 7 mmol) in dioxane (15 mL) was added dropwise. After stirring for 4 h at 0° C. and for 8 h at rt (reaction monitored by TLC eluting with n-Hexane/1-PrOH 1/1, Rf of compound B=0.36), the mixture was concentrated in vacuo. The residual aqueous solution was extracted with AcOEt (3×15 mL), then the combined organic extracts dried over Na₂SO₄ and concentrated in vacuum. Purification by flash chromatography (gradient n-Hexane/AcOEt 90/10 to n-Hexane/Ethyl acetate 20/80) gave product B. (1.1 g, 34%).

### C) (4S-trans)-4-(amino-N-ethyl-N-Fmoc)-5,6-dihydro-6-methyl-4H-thieno[2,3-b]thiopyran-2-sulfonylamido-7,7-dioxide

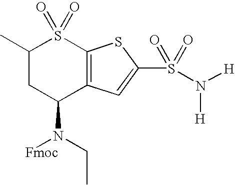

A solution of compound B (1.76 g, 3 mmol) in MeOH (30 ml) and a 37% aqueous of HCl (12 ml) was stirred for 12 h at 50° C. and for 2 h at reflux and then the mixture was concentrated in vacuum. The residue was dissolved in CH₂Cl₂ (20 mL) and washed with brine (2×10 mL). The combined CH₂Cl₂ extracts were dried over Na₂SO₄ and concentrated in vacuo. Purification by flash chromatography (n-hexane/acetone 6/4) gave product C as a white solid (0.9 g, 55%). (TLC eluting with CH₂Cl₂/MeOH 95/5, Rf=0.58)

### D) 4S-trans)-4-(amino-N-ethyl-N-Fmoc)-5,6-dihydro-6-methyl-4H-thieno[2,3-b]thiopyran-2-sulfonylamido-N-8(6-(nitrooxy)hexanoyl)-7,7-dioxide

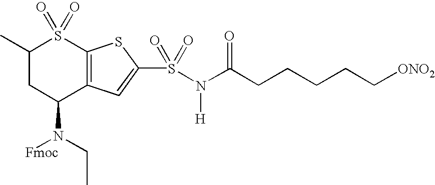

To a solution of compound C (0.899 g, 1.64 mmol) in CH₂Cl₂ (10 ml), DMAP (200 mg, 1.64 mmol) and scandium triflate (162 mg, 0.33 mmol) were added. The mixture was cooled to 0° C. and a solution of 6-(nitrooxy)hexanoate pentafluorophenyl ester (0.596 mg, 1.4 mmol) in CH₂Cl₂ (5 mL) was added dropwise. After stirring for 24 h at rt the reaction was cooled to 0° C. using an iced water bath and water (20 mL) was added. The aqueous solution was extracted with CH₂Cl₂ (3×10 mL). The combined CH₂Cl₂ extracts were dried over Na₂SO₄ and concentrated in vacuo. Purification by flash chromatography (eluent n-Hexane/Acetone 50/50) gave product D. (0.634 g, 55%).

### E) 4S-trans)-4-(amino-N-ethyl)-5,6-dihydro-6-methyl-4H-thieno[2,3-b]thiopyran-2-sulfonylamido-N-8(6-(nitrooxy)hexanoyl)-7,7-dioxide

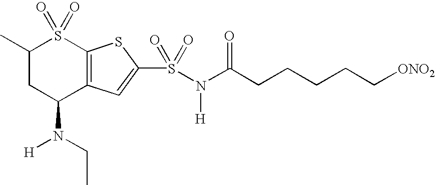

To a solution of compound D (0.634 g, 0.9 mmol) in acetonitrile (10 ml), morpholine (0.439 mL, 4.5 mmol) was added. After stirring for 3 h at rt the reaction was concentrated in vacuum. The residue was dissolved in ethyl acetate (20 mL) and washed a solution of NaH₂PO₄ (2×10 mL). The combined organic extracts were dried over Na₂SO₄ and concentrated in vacuo. Purification by flash chromatography (gradient CH₂Cl₂ 100% to CH₂Cl₂/MeOH 94/6) gave product E. (0.125 g, 37%).

¹H-NMR (DMSO-d₆) δ: 7.40 (1H, s); 4.47 (2H, t); 4.00-3.80 (2H, m); 2.81-2.52 (2H, m); 2.45-2.20 (2H, m); 2.00 (2H, t); 1.55 (2H, m); 1.50-1.40 (2H, m); 1.33 (3H, d), 1.32-1.20 (2H, m); 1.05 (3H, t).

### EXAMPLE 2

### Synthesis of Compound of Formula (62)

### (4S-trans)-4-(amino-N-ethyl-N-(2,3-bis(nitrooxy) propyloxycarbonyl)-5,6-dihydro-6-methyl-4H-thieno[2,3-b]thiopyran-2-sulfonylamido-7,7-dioxide

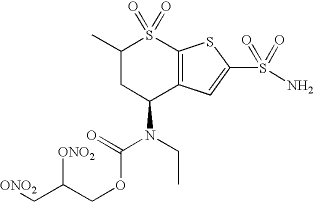

### F) (4S-trans)-4-(amino-N-ethyl-N-(2,3-propenyloxycarbonyl)-5,6-dihydro-6-methyl-4H-thieno[2,3-b]thiopyran-2-sulfonylamido-N-(dimethylamino methylen)-7,7-dioxide

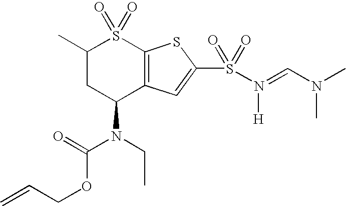

Compound A (2.65 g, 7 mmol) was dissolved in CH₂Cl₂ (30 mL) and cooled to 0° C., then pyridine (0.56 mL, 7 mmol), DMAP (33 mg, 0.27 mmol) and a solution of AllocCl (1.27 g, 10.5 mmol in 30 ml of CH₂Cl₂) were sequentially added.

After stirring overnight at rt, the reaction mixture was poured into water (20 mL) and extracted with CH₂Cl₂ (3×10 mL). The combined CH₂Cl₂ extracts were washed with water (20 mL), dried over Na₂SO₄ and concentrated in vacuo. Purification by flash chromatography (n-Hexane/Acetone 1/1 Rf=0.26) gave product F as a white solid (1.1 g, 34%).

### G) (4S-trans)-4-(amino-N-ethyl-N-(2,3-propenyloxycarbonyl)-5,6-dihydro-6-methyl-4H-thieno[2,3-b]thiopyran-2-sulfonylamido-7,7-dioxide

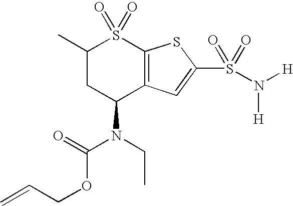

Compound F (1.1 g, 2.37 mmol) was dissolved in MeOH (24 mL) and a 37% aqueous solution of HCl (9.5 ml). The reaction was monitored by TLC eluting with CH₂Cl₂/MeOH 95/5 (Rf compound G=0.26); after stirring for 12 h at 50° C. and for 2 h at reflux, the mixture was concentrated in vacuo. The residue was dissolved in CH₂Cl₂ (20 mL), washed with a saturated solution of NaHCO₃ (10 mL) and brine (2×10 mL). The CH₂Cl₂ extracts were dried over Na₂SO₄ and concentrated to give the product G (530 mg, 55%).

### H) (4S-trans)-4-(amino-N-ethyl-N-(2,3-bis(nitrooxy)propyloxycarbonyl)-5,6-dihydro-6-methyl-4H-thieno[2,3-b]thiopyran-2-sulfonylamido-7,7-dioxide

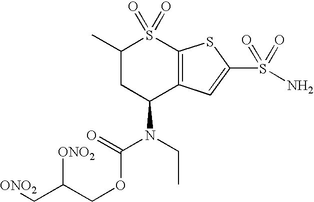

To a solution of compound G (0.265 g, 0.64 mmol) in acetonitrile (11 mL), iodine (0.165 g, 0.649 mmol) and silver nitrate (331 mg, 1.95 mmol) were added. After stirring for 20 h at 70° C. the mixture was concentrated in vacuo. Purification by flash chromatography (n-Hexane/Ethyl acetate 1/1) gave mixture of mononitrate and dinitrate derivative (0.138 g). The mixture was dissolved in acetonitrile (1.4 mL) and silver nitrate (0.098 g) was added. The reaction was performed using the microwave at 120° c. for 20 min. The mixture was concentrated in vacuo. Purification by flash chromatography (n-Hexane/Ethyl acetate 1/1) gave product H as a white solid (0.066 g, 20%).

¹H-NMR (CDCl₃) δ: 7.39 (1H, s); 5.48-5.32 (3H, m); 5.30-5.21 (1H, m); 4.91-4.84 (1H, m); 4.80-4.56 (2H, m); 3.75-3.50 (1H, m); 3.45-3.25 (1H, m); 3.3.20-3.08 (1H, m); 3.00-2.85 (1H, m); 2.61-2.45 (1H, m); 1.53 (3H, d); 1.30-1.17 (3H, dt).

### EXAMPLE 3

### Synthesis of Compound of Formula (72)

### (4S-trans)-4-(amino-N-ethyl-N-(4-(nitrooxy)butyloxycarbonyl)-5,6-dihydro-6-methyl-4H-thieno[2,3-b]thiopyran-2-sulfonylamido-7,7-dioxide

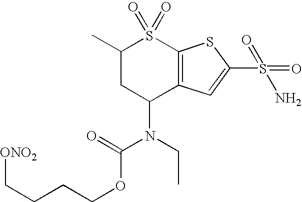

### I) (4S-trans)-4-(amino-N-ethyl-N-(4-chlorobutyloxycarbonyl)-5,6-dihydro-6-methyl-4H-thieno[2,3-b]thiopyran-2-sulfonylamido-N-(dimethylamino methylen)-7,7-dioxide

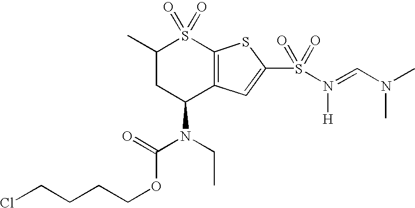

To a solution of compound A (0.4 g, 1.04 mmol) in CH₂Cl₂ (8 mL) cooled to 0° C., triethylamine (0.29 mL, 2.1 mmol) and 4-chlorobutylchloroformate (0.28 mL, 2.1 mmol) were sequentially added.

After stirring for 17 hours at room temperature triethylamine (0.29 mL, 2.1 mmol) and 4-chlorobutylchloroformate (0.28 mL, 2.1 mmol) were added. After stirring for 8 hours at room temperature triethylamine (0.29 mL, 2.1 mmol) and 4-chlorobutylchloroformate (0.28 mL, 2.1 mmol) were added.

After stirring for 24 hours at room temperature, the reaction mixture was poured into water (20 mL) and extracted with CH₂Cl₂ (3×10 mL). The combined organic extracts were washed with water (20 mL), dried over Na₂SO₄ and concentrated in vacuo. Purification by flash chromatography (AcOEt) gave product I as a pale yellow gum (0.32 g, 68%).

### L) (4S-trans)-4-(amino-N-ethyl-N-(4-chlorobutyloxycarbonyl)-5,6-dihydro-6-methyl-4H-thieno[2,3-b]thiopyran-2-sulfonylamido-7,7-dioxide

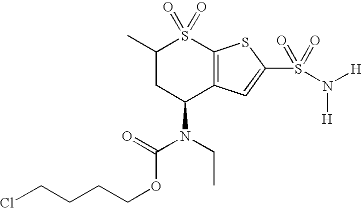

To compound I (0.096 g, 0.18 mmol) in tetrahydrofurane (1.8 mL) was added a 37% aqueous solution of HCl (0.74 mL). The mixture was stirred in microwave at 110° C. and 3 bar for 28 minutes (the reaction was monitored by TLC eluting with CH₂Cl₂/MeOH 90/10, Rf compound L=0.5), the mixture was concentrated in vacuo and the residue was dissolved in CH₂Cl₂ (20 mL), washed with a saturated solution of NaHCO₃ (10 mL) and brine (2×10 mL). The organic layer was dried over Na₂SO₄ and concentrated in vacuo. Purification by flash chromatography (AcOEt) gave product L as an oil (0.058 g, 68%).

### M) (4S-trans)-4-(amino-N-ethyl-N-(4-iodobutyloxycarbonyl)-5,6-dihydro-6-methyl-4H-thieno[2,3-b]thiopyran-2-sulfonylamido-7,7-dioxide

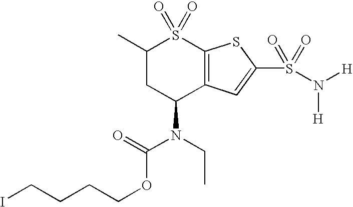

To a solution of compound L (0.058 g, 0.126 mmol) in acetonitrile (3 mL) sodium iodide (0.094 g, 0.63 mmol) was added. The reaction was stirred in microwave at 150° C. for 30 minutes. The salts were filtered off and the mixture was concentrated in vacuo to give the compound M (0.060 mg) as a yellow solid.

### (4S-trans)-4-(amino-N-ethyl-N-(4-(nitrooxy)butyloxycarbonyl)-5,6-dihydro-6-methyl-4H-thieno[2,3-b]thiopyran-2-sulfonylamido-7,7-dioxide

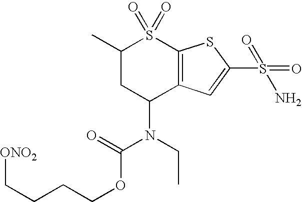

To a solution of compound M (0.060 g, 0.108 mmol) in acetonitrile (3 mL) silver nitrate (0.086 g, 0.50 mmol) was added. The reaction was stirred in microwave at 130° C. for 4 minutes. The salts were filtered off and the mixture was concentrated in vacuo.

Purification by flash chromatography (AcOEt) gave (4S-trans)-4-(amino-N-ethyl-N-(4-(nitrooxy)butyloxycarbonyl)-5,6-dihydro-6-methyl-4H-thieno[2,3-b]thiopyran-2-sulfonylamido-7,7-dioxide as a white foam (0.053 g, 87%).

¹H-NMR (CDCl₃) δ: 7.38 (1H, s); 5.55 (2H, s); 5.3 (1H, m); 4.52 (2H, m); 4.20-4.15 (2H, m); 3.67 (1H, m); 3.31-3.06 (2H, m); 3.00-2.85 (1H, m); 2.50-2.45 (1H, m); 1.85 (4H, m); 1.53 (3H, d); 1.20-1.10 (3H, dt).

### EXAMPLE 4

### Synthesis of Compound of Formula (65)

### (4S-trans)-4-(amino-N-ethyl-N-(4-(nitrooxymethyl)benzoyl)-5,6-dihydro-6-methyl-4H-thieno[2,3-b]thiopyran-2-sulfonylamido-7,7-dioxide

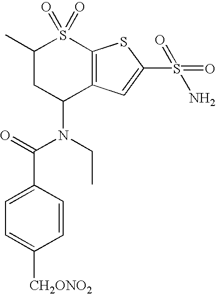

### N) (4S-trans)-4-(amino-N-ethyl-N-(4-(chloromethyl)benzoyl)-5,6-dihydro-6-methyl-4H-thieno[2,3-b]thiopyran-2-sulfonylamido-N-(dimethylamino methylen)-7,7-dioxide

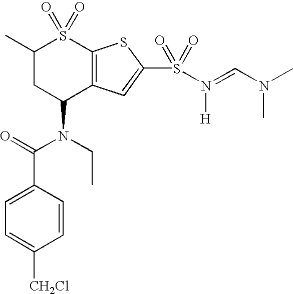

To a solution of compound A (0.1 g, 0.26 mmol) in CH₂Cl₂ (2 mL) cooled to 0° C., triethylamine (0.07 mL, 0.52 mmol) and 4-chloromethylbenzylchloride (0.1 g, 0.52 mmol) were sequentially added.

The reaction mixture was stirred at room temperature for 70 hours and then it was poured into water (20 mL) and extracted with CH₂Cl₂ (3×10 mL). The combined organic extracts were washed with water (20 mL), dried over Na₂SO₄ and concentrated in vacuo. Purification by Biotage flash chromatography (eluent AcOEt/n-hexane 1/1 (250 mL) and AcOEt (500 mL)) gave product N as a foam (0.12 g, 86%).

### O) (4S-trans)-4-(amino-N-ethyl-N-(4-(chloromethyl)benzoyl)-5,6-dihydro-6-methyl-4H-thieno[2,3-b]thiopyran-2-sulfonylamido-7,7-dioxide

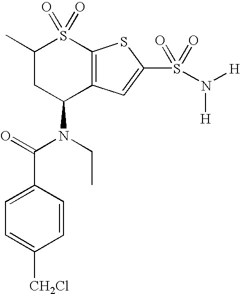

To a solution of compound N (0.12 g, 0.22 mmol) in tetrahydrofurane (2.1 mL), a 37% aqueous solution of HCl (0.90 mL) was added and the mixture was stirred in microwave at 110° C. and 3 bar for 28 minutes (the reaction was monitored by TLC eluting with AcOEt, Rf compound O=0.26). The mixture was concentrated in vacuo and the residue was dissolved in CH₂Cl₂ (20 mL), washed with a saturated solution of NaHCO₃ (10 mL) and brine (2×10 mL). The organic layer was dried over Na₂SO₄ and concentrated in vacuo. Purification by Biotage flash chromatography (eluent AcOEt/n-hexane 1/1 (250 mL) and AcOEt (500 mL)) gave product O (0.09 g, 84%).

### (4S-trans)-4-(amino-N-ethyl-N-(4-(nitrooxymethyl)benzoyl)-5,6-dihydro-6-methyl-4H-thieno[2,3-b]thiopyran-2-sulfonylamido-7,7-dioxide

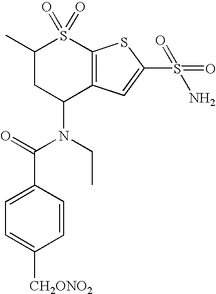

To a solution of compound O (0.09 g, 0.189 mmol) in acetonitrile (4.5 mL) silver nitrate (0.129 g, 0.76 mmol) was added. The reaction was stirred in microwave at 110° C. for 20 minutes. The salts were filtered off and the mixture was concentrated in vacuo.

Purification by Biotage flash chromatography (eluent AcOEt/n-hexane 1/1 (250 mL) and AcOEt (500 mL)) gave (4S-trans)-4-(amino-N-ethyl-N-(4-(nitrooxymethyl)benzoyl)-5,6-dihydro-6-methyl-4H-thieno[2,3-b]thiopyran-2-sulfonylamido-7,7-dioxide as a white foam (0.063 g, 66%).

¹H-NMR (CDCl₃) δ: 7.52-7.42 (5H, m); 5.49 (2H, s); 5.3t (1H, m); 3.81 (1H, m); 3.65-3.44 (2H, m); 3.10-2.95 (1H, m); 2.70-2.50 (1H, m); 1.55 (3H, d); 1.20-1.10 (3H, dt).

### EXAMPLE 5

### Synthesis of Compound of Formula (89)

### (4S-trans)-4-(amino-N-ethyl-N-(6-(nitrooxy)hexanoyl)-5,6-dihydro-6-methyl-4H-thieno[2,3-b]thiopyran-2-sulfonylamido-7,7-dioxide

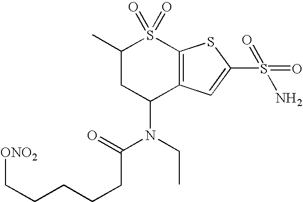

### P) (4S-trans)-4-(amino-N-ethyl-N-(6-bromohexanoyl)-5,6-dihydro-6-methyl-4H-thieno[2,3-b]thiopyran-2-sulfonylannido-N-(dimethylamino methylen)-7,7-dioxide

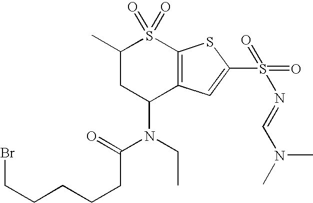

To a solution of compound A (0.2 g, 0.53 mmol) in CH₂Cl₂ (4 mL) cooled to 0° C., triethylamine (0.15 mL, 1.08 mmol) and 6-bromocaproylchloride (0.16 g, 1.08 mmol) were sequentially added. Allowed to stir for 68 hours at room temperature, the reaction mixture was poured into water (20 mL) and extracted with CH₂Cl₂ (3×10 mL). The combined organic extracts were washed with water (20 mL), dried over Na₂SO₄ and concentrated in vacuo. Purification by Biotage flash chromatography (eluent AcOEt/n-hexane 1/1 (450 mL) and AcOEt (500 mL)) gave product P as a white foam (0.25 g, 82%).

### Q) (4S-trans)-4-(amino-N-ethyl-N-(6-(nitrooxy)hexanoyl)-5,6-dihydro-6-methyl-4H-thieno[2,3-b]thiopyran-2-sulfonylamido-N-(dimethylamino methylen)-7,7-dioxide

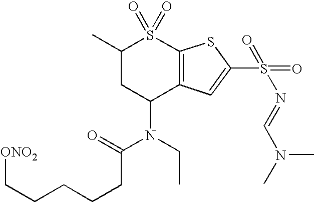

To a solution of compound P (0.37 g, 0.66 mmol) in acetonitrile (16 mL) silver nitrate (0.456 g, 2.65 mmol) was added. The reaction was stirred in microwave at 110° C. for 20 minutes. The salts were filtered off and the mixture was concentrated in vacuo. Purification by Biotage flash chromatography (eluent AcOEt) gave product Q as a white foam (0.253 g, 71%).

### (4S-trans)-4-(amino-N-ethyl-N-(6-(nitrooxy)hexanoyl)-5,6-dihydro-6-methyl-4H-thieno[2,3-b]thiopyran-2-sulfonylamido-7,7-dioxide

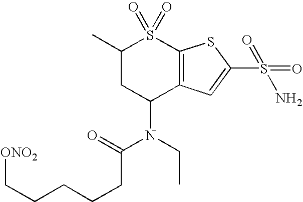

To a solution of compound Q (0.25 g, 0.47 mmol) in tetrahydrofurane (4.5 mL) a 37% aqueous solution of HCl (1.87 mL) was added. The mixture was stirred in microwave at 110° C. and 3 bar for 28 minutes (the reaction was monitored by TLC eluting with AcOEt, Rf of the title compound=0.36). The mixture was concentrated in vacuo and the residue was dissolved in CH₂Cl₂ (20 mL), washed with a saturated solution of NaHCO₃ (10 mL) and brine (2×10 mL). The organic layer was dried over Na₂SO₄ and concentrated in vacuo.

Purification by Biotage flash chromatography (eluent AcOEt/n-hexane 1/1 (250 mL) and AcOEt (500 mL)) gave (4S-trans)-4-(amino-N-ethyl-N-(6-(nitrooxy)hexanoyl)-5,6-dihydro-6-methyl-4H-thieno[2,3-b]thiopyran-2-sulfonylamido-7,7-dioxide (0.17 g, 75%).

¹H-NMR (CDCl₃) δ: 7.20 (1H, s); 5.49 (1H, m); 4.50 (2H, t); 3.71 (1H, m); 3.41 (1H, m); 3.23 (1H, m); 2.86 (1H, m); 2.50 (3H, m); 1.90-1.55 (6H, m); 1.60 (3H, m); 1.40-1.20 (3H, m).

### EXAMPLE 6

### Synthesis of Compound of Formula (90)

### (4S-trans)-4-(amino-N-ethyl-N-(3-(nitrooxy) propyloxycarbonyl)-5,6-dihydro-6-methyl-4H-thieno[2,3-b]thiopyran-2-sulfonylamido-7,7-dioxide

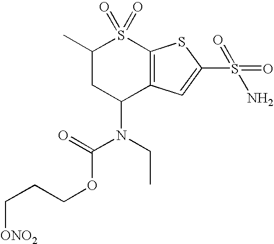

### R) (4S-trans)-4-(amino-N-ethyl-N-(3-chloropropyloxycarbonyl)-5,6-dihydro-6-methyl-4H-thieno[2,3-b]thiopyran-2-sulfonylamido-N-(dimethylamino methylen)-7,7-dioxide

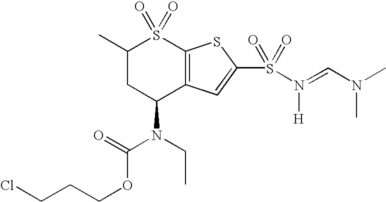

To a solution of compound A (0.41 g, 1.08 mmol) in CH₂Cl₂ (8 mL) cooled to 0° C., triethylamine (0.30 mL, 2.16 mmol) and 3-chloropropylchloroformate (0.26 mL, 2.16 mmol) were sequentially added.

After stirring for 24 hours at room temperature triethylamine (0.30 mL, 2.16 mmol) and 3-chloropropylchloroformate (0.26 mL, 2.16 mmol) were added. After stirring for 4 hours at room temperature triethylamine (0.30 mL, 2.16 mmol) and 3-chloropropylchloroformate (0.26 mL, 2.16 mmol) were added.

After stirring for 2 hours at room temperature, the reaction mixture was poured into water (20 mL) and extracted with CH₂Cl₂ (3×10 mL). The combined organic extracts were washed with water (20 mL), dried over Na₂SO₄ and concentrated in vacuo.

Purification by Biotage flash chromatography (eluent AcOEt/n-hexane 1/1, AcoEt) gave product R as a white foam (0.38 g, 72%).

### S) (4S-trans)-4-(amino-N-ethyl-N-(3-iodopropyloxycarbonyl)-5,6-dihydro-6-methyl-4H-thieno[2,3-b]thiopyran-2-sulfonylamido-N-(dimethylamino methylen)-7,7-dioxide

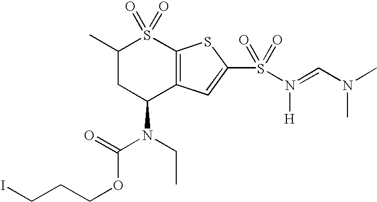

To a solution of compound R (0.385 g, 0.77 mmol) in acetonitrile (15 mL) sodium iodide (0.577 g, 3.85 mmol) was added. The reaction mixture was stirred in microwave at 150° C. for 30 minutes. The salts were filtered off and the mixture was concentrated in vacuo to give the compound S (0.450 mg).

### T) (4S-trans)-4-(amino-N-ethyl-N-(3-(nitrooxy)propyloxycarbonyl)-5,6-dihydro-6-methyl-4H-thieno[2,3-b]thiopyran-2-sulfonylamido-N-(dimethylamino methylen)-7,7-dioxide

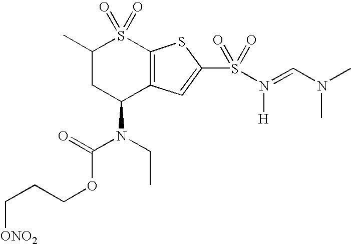

To a solution of compound S (0.45 g, 0.77 mmol) in acetonitrile (15 mL) silver nitrate (0.525 g, 3.08 mmol) was added. The reaction mixture was stirred in microwave at 110° C. for 20 minutes. The salts were filtered off and the mixture was concentrated in vacuo. Purification by Biotage flash chromatography (eluent AcOEt/n-hexane 1/1, AcOEt) gave the product T (0.303 g, 75%).

### (4S-trans)-4-(amino-N-ethyl-N-(3-(nitrooxy)propyloxycarbonyl)-5,6-dihydro-6-methyl-4H-thieno[2,3-b]thiopyran-2-sulfonylamido-7,7-dioxide

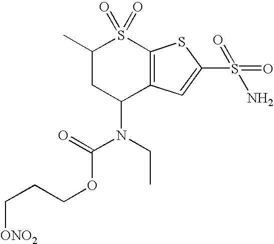

Compound S (0.30 g, 0.57 mmol) was dissolved in tetrahydrofurane (5.5 mL) and a 37% aqueous solution of HCl (2.29 mL). The mixture was stirred in microwave at 110° C. and 3 bar for 28 minutes (the reaction was monitored by TLC eluting with AcOEt, Rf compound L=0.23). The mixture was concentrated in vacuo and the residue was dissolved in CH₂Cl₂ (20 mL), washed with a saturated solution of NaHCO₃ (10 mL) and brine (2×10 mL). The organic layer was dried over Na₂SO₄ and concentrated in vacuo. Purification by flash chromatography (eluent AcOEt/n-hexane 1/1, AcOEt) gave (4S-trans)-4-(amino-N-ethyl-N-(3-(nitrooxy)propyloxycarbonyl)-5,6-dihydro-6-methyl-4H-thieno[2,3-b]thiopyran-2-sulfonylamido-7,7-dioxide (0.184 g, 68%).

¹H-NMR (CDCl₃) δ: 7.40 (1H, s); 5.39-5.25 (3H, m); 4.62 (2H, m); 4.31 (2H, m); 3.67 (1H, m); 3.49-3.30 (1H, m); 3.10-3.00 (1H, m); 3.00-2.83 (1H, m); 2.51-2.46 (1H, m); 2.17 (2H, m); 1.53 (3H, d); 1.19 (3H, dt).

### EXAMPLE 7

### Synthesis of Compound of Formula (37)

### (4S-trans)-4-(ethylamino)-5,6-dihydro-6-methyl-4H-thieno[2,3-b]thiopyran-2-sulfonylamido-N-(4-(nitrooxy)butyloxycarbonyl)-7,7-dioxide

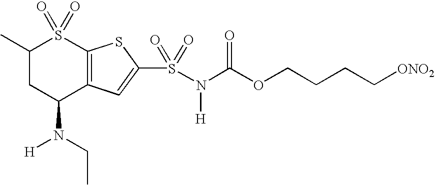

### T) 4S-trans)-4-(amino-N-ethyl-N-Fmoc)-5,6-dihydro-6-methyl-4H-thieno[2,3-b]thiopyran-2-sulfonylamido-N-(4-(nitrooxy)butyloxy carbonyl)-7,7-dioxide

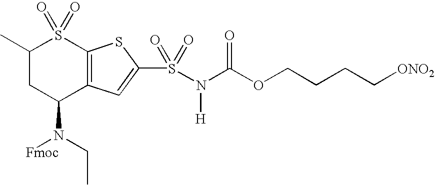

To a solution of compound C (0.800 g, 1.46 mmol) in CH₂Cl₂ (10 ml), DMAP (178 mg, 1.46 mmol) and scandium triflate (144 mg, 0.29 mmol) were added. The mixture was cooled to 0° C. and a solution of 4-(nitrooxy)butyl pentafluorophenyl carbonate (0.345 mg, 1.46 mmol) in CH₂Cl₂ (5 mL) was added dropwise. After stirring for 12 h at rt the reaction was cooled to 0° C. using an iced water bath and water (20 mL) was added. The aqueous solution was extracted with CH₂Cl₂ (3×10 mL). The combined CH₂Cl₂ extracts were dried over Na₂SO₄ and concentrated in vacuo. Purification by flash chromatography (eluent CH₂Cl₂/MeOH 98/2) gave product T. (0.360 g, 34%).

### (4S-trans)-4-(ethylamino)-5,6-dihydro-6-methyl-4H-thieno[2,3-b]thiopyran-2-sulfonylamido-N(4-(nitrooxy)butyloxycarbonyl)-7,7-dioxide

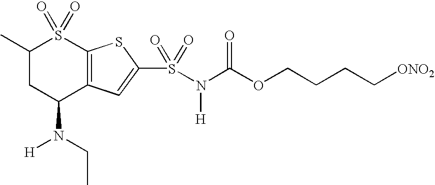

To a solution of compound T (0.360 g, 0.51 mmol) in acetonitrile (3.6 ml), morpholine (0.248 mL, 2.5 mmol) was added. After stirring for 3 h at rt the reaction was concentrated in vacuum. The residue was dissolved in ethyl acetate (20 mL) and washed a solution of NaH₂PO₄ (2×10 mL). The combined organic extracts were dried over Na₂SO₄ and concentrated in vacuo. Purification by flash chromatography (eluent CH₂Cl₂/MeOH 98/2) gave the desired product. (0.098 g, 40%).

¹H-NMR (DMSO-d₆) δ: 7.45 (1H, s); 4.50 (2H, t); 4.40 (1H, m); 4.00-3.80 (1H, m); 3.75 (2H, m); 3.15-3.00 (1H, m); 3.00-2.80 (1H, m); 2.50-2.40 (2H, m); 1.65 (2H, m); 1.55 (2H, m); 1.33 (3H, d); 1.15 (3H, t).

### Evaluation of the Carbonic Anhydrase Inhibition Against Isoenzymes Ca I, II and IV.

The tested compounds were:
Dorzolamide
Compound of ex. 1: (4S-trans)-4-(ethylamino)-5,6-dihydro-6-methyl-4H-thieno[2,3-b]thiopyran-2-sulfonylamido-N((6-nitrooxy)hexanoyl)-7,7-dioxide (of compound of formula (33));
Compound of ex. 2: (4S-trans)-4-(amino-N-ethyl-N-(2,3-bis(nitrooxy)propyloxycarbonyl)-5,6-dihydro-6-methyl-4H-thieno[2,3-b]thiopyran-2-sulfonylamido-7,7-dioxide (compound of formula (62));
Compound of ex. 3: (4S-trans)-4-(amino-N-ethyl-N-(4-(nitrooxy)butyloxycarbonyl)-5,6-dihydro-6-methyl-4H-thieno[2,3-b]thiopyran-2-sulfonylamido-7,7-dioxide (compound of formula (72));
Compound of ex. 4: (4S-trans)-4-(amino-N-ethyl-N-(4-(nitrooxymethyl)benzoyl)-5,6-dihydro-6-methyl-4H-thieno[2,3-b]thiopyran-2-sulfonylamido-7,7-dioxide (compound of formula (65));
Compound of ex. 5: (4S-trans)-4-(amino-N-ethyl-N-(6-(nitrooxy)hexanoyl)-5,6-dihydro-6-methyl-4H-thieno[2,3-b]thiopyran-2-sulfonylamido-7,7-dioxide (compound of formula (89));
Compound of ex. 6: (4S-trans)-4-(amino-N-ethyl-N-(3-(nitrooxy)propyloxycarbonyl)-5,6-dihydro-6-methyl-4H-thieno[2,3-b]thiopyran-2-sulfonylamido-7,7-dioxide (compound of formula (90));
Compound of ex. 7: (4S-trans)-4-(ethylamino)-5,6-dihydro-6-methyl-4H-thieno[2,3-b]thiopyran-2-sulfonylamido-N(4-(nitrooxy)butyloxycarbonyl)-7,7-dioxide (compound of formula (37)).

### Experimental Procedure

An SX.18MV-R Applied Photophysics stopped flow instrument was used for assaying the CA CO₂ hydration activity. Phenol red (0.2 mM) was used as indicator (pH 6.8-8.4), working at the absorbance maximum of 557 nm. The buffer solution was constituted by 10 mM HEPES, 0.1 M Na₂SO₄, and TRIZMA hydrochloride 0.01 M, adjusting the pH solution at 7.5 with NaOH (0.1 M). The CA-catalyzed CO₂ hydration reaction was followed for a period of 1-20 s, depending on the isoform used. Satured CO₂ solution in bidistilled water at 20° C. was used as substrate.

Stock solutions of inhibitor (1 mM) were prepared with buffer solution with 10-20% (v/v) DMSO, and dilutions up to 0.1 nM. To allow for the formation of the E-1 complex, inhibitor and enzyme solutions were preincubated during 15 min at room temperature prior to assay. Enzyme concentration was 0.1 μM for CA I and II, 10 μM for CA IV. The human CA I and CA II are commercially available, whereas hCA IV (tumor) is cloned isoform.

Each experiment was done in triplicate.

| Compound | hCA I | hCA II | H CA IV |
|---|---|---|---|
| Dorzolamide | 50000 | 9 | — |
| Ex. 1 | 15000 | 251 | 3404 |
| Ex. 2 | 1520 | 63 | 3905 |
| Ex. 3 | 705 | 76 | 339 |
| Ex. 4 | 470 | 71 | 46 |
| Ex. 5 | 2950 | 14 | 4360 |
| Ex. 6 | 410 | 13 | 181 |
| Ex. 7 | 42300 | 886 | 411 |

### Test on Vascular Tone

The ability of the nitroderivatives of carbonic anhydrase inhibitors to induce vasorelaxation in comparison to native CAI, was tested in vitro in isolated rabbit thoracic aorta preparations (Wanstall J. C. et al., Br. J. Pharmacol., 134:463-472, 2001). Male New Zealand rabbits were anaesthetized with thiopental-Na (50 mg/kg, iv), sacrificed by exsanguinations and then the thorax was opened and the aorta dissected. Aortic ring preparations (4 mm in length) were set up in physiological salt solution (PSS) at 37° C. in small organ chambers (5 ml). The composition of PSS was (mM): NaCl 130, NaHCO₃ 14.9, KH₂PO₄ 1.2, MgSO₄ 1.2, HEPES 10, CaCl₂ ascorbic acid 170 and glucose 1.1 (95% O₂/5% CO₂; pH 7.4). Each ring was mounted under 2 g passive tension. Isometric tension was recorded with a Grass transducer (Grass FT03) attached to a BIOPAC MP150 System. Preparations were allowed to equilibrate for 1 h, and then contracted submaximally with noradrenaline (NA, 1 μM) and, when the contraction was stable, acetylcholine (ACh, 10 μM) was added. A relaxant response to ACh indicated the presence of a functional endothelium. Vessels that were unable to contract NA or showed no relaxation to Ach were discarded. When a stable precontraction was reached, a cumulative concentration-response curve to either of the vasorelaxant agents was obtained in the presence of a functional endothelium. Each arterial ring was exposed to only one combination of inhibitor and vasorelaxant. Moreover, the effect of the soluble guanylyl cyclase inhibitor ODQ (1-H-(1,2,4)-oxadiazol(4,3-a)quinoxalin-1-one) on vasorelaxation elicited by the compounds was examined preincubating the aortic rings with ODQ (10 μM) for 20 min.

Responses to relaxing agents are expressed as a percentage of residual contraction and plotted against concentration of test compound. EC₅₀ values (where EC₅₀ is the concentration producing 50% of the maximum relaxation to the test compound) were interpolated from these plots.

During the experimental period, the plateau obtained with NA was stable without significant spontaneous loss of contraction in the aortic rings. Under these experimental conditions, the carbonic anhydrase inhibitors did not produce relaxation at any of the concentration tested, the curve being not different from that built up in the presence of vehicle alone.

The nitroderivatives of the invention have EC₅₀ values in the range of 1-50 μM. Furthermore, in experiments performed in the presence of ODQ (10 μM), the vasorelaxant responses to tested compounds were inhibited.

1. A method for treating eye disorders in a patient in need thereof comprising administering a therapeutically effective amount of a carbonic anhydrase inhibitor able to release nitric oxide.

2. The method of claim 1, wherein the eye disorder is glaucoma, ocular hypertension, age-related macular degeneration, diabetic macular edema, diabetic retinopathy, hypertensive retinopathy and retinal vasculopathies.

3. A method of claim 1 wherein carbonic anhydrase inhibitor is a compound having an inhibition constant (K₁) against the isoenzyme CAII in the range of 0.01-200 nM.

4. A method of claim 1 wherein the carbonic anhydrase inhibitor able to release nitric oxide is a compound having an EC₅₀ value in the range of 1-50 μM.

5. A compound of general formula (I) or a pharmaceutically acceptable salt or stereoisomer thereof.
R—(X—Y—ONO₂)ₘ (I)
wherein:
m is an integer equal to 1 or 2;
R is: 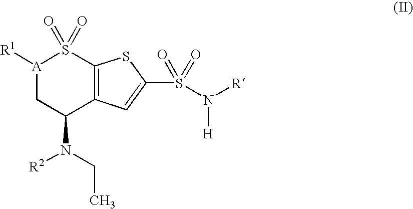
wherein
R¹ is —CH₃ or —(CH₂)₃—OCH₃;
R² is H or a group —(X—Y—ONO₂),
R′ is H or a group —(X—Y—ONO₂);
with the proviso that at least one of R² or R′ is a —(X—Y—ONO₂) group;
A is a carbon or nitrogen atom;
X is —CO—, —COO—;
Y is a bivalent radical having the following meaning:
(a)
straight or branched Cₗ-C₂₀ alkylene,
straight or branched C₁-C₂₀ alkylene substituted with one or more of the substituents selected from the group consisting of: halogen atoms, hydroxy, —ONO₂ or T, wherein T is —OC(O)(C,-C₁₀ alkyl)-ONO₂ or —O(C₁-Cₗo alkyl)-ONO₂;
cycloalkylene with 5 to 7 carbon atoms into cycloalkylene ring, the ring being optionally substituted with side chains T₁, wherein T₁ is straight or branched C₁-C_{1,3} alkyl; 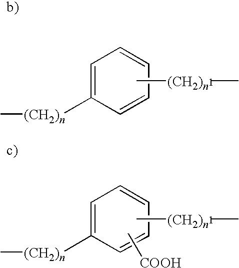
wherein n is an integer from 0 to 20, and n¹ is an integer from 1 to 20; 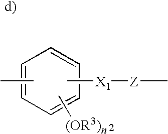
wherein
X₁=—OCO— or —COO—;
Z is —(CH₂)ₙ¹— or the bivalent radical defined above under b);
n¹ is as defined above and
n² is an integer from 0 to 2 and R³ is H or —CH₃; 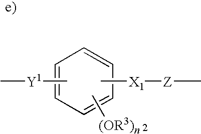
wherein:
Y¹ is —CH₂—CH₂—CH₂)ₙ^{2a} or —CH═CH—(CH₂)ₙ^{2a} wherein n^{2a} is from 0 to 2;
Z, n¹, n², R³ and X₁ are as defined above; 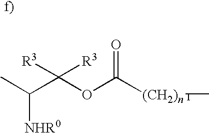
wherein:
n¹ is an integer from 1 to 20 and R³ is H or —CH₃,
R⁰ is H or —COCH₃;
with the proviso that when Y is selected from the bivalent radicals mentioned under b)-f), then the terminal —ONO₂ group is bound to —(CH₂)ₙ¹, 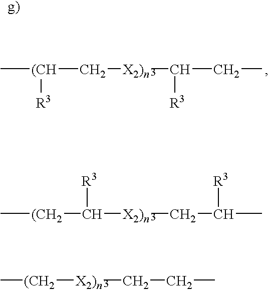
wherein X₂ is -0- or —S—, n³ is an integer from 1 to 6, R³ is H or —CH₃; 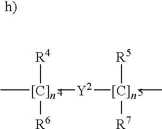
wherein:
n⁴ is an integer from 0 to 10;
n⁵ is an integer from 1 to 10;
R⁴, R⁵, R⁶, R⁷ are the same or different, and are H or straight or branched C₁-C₄ alkyl;
wherein the —ONO₂ group is linked to 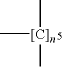
wherein n⁵ is as defined above;
Y² is an heterocyclic saturated, unsaturated or aromatic 5 or 6 members ring, containing one or more heteroatoms selected from nitrogen, oxygen, sulfur,
and is selected from the group consisting of:

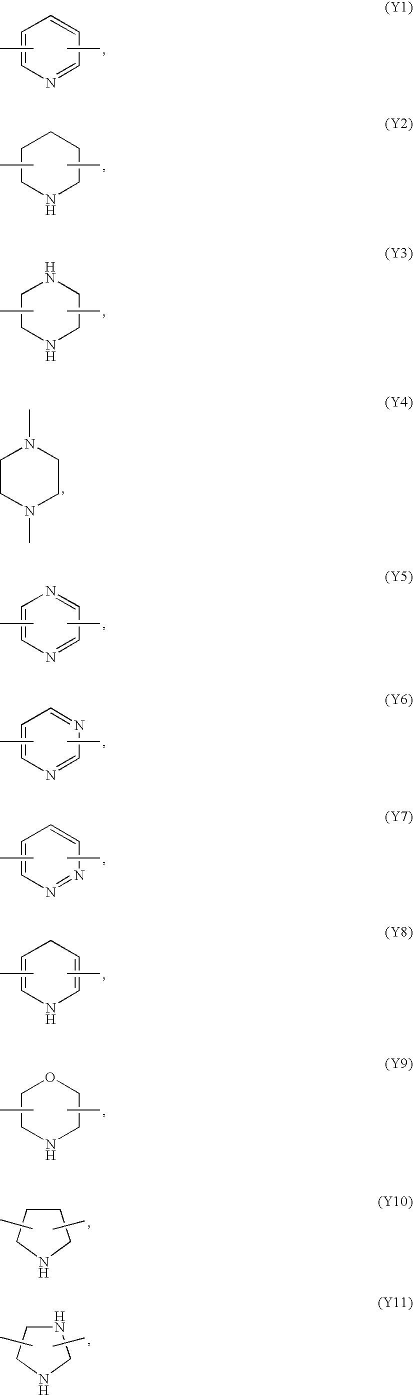

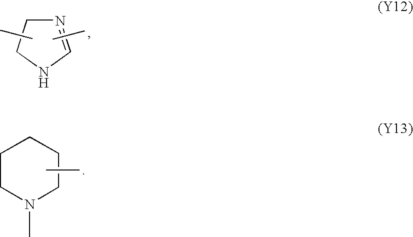

6. A compound and pharmaceutically acceptable salts or stereoisomers thereof according to claim 5 wherein:
m is 1 and
in R of formula (II):
A is a carbon atom,
R¹ is —CH₃,
R′ is an hydrogen atom and
R² is the group —(X—Y—ONO₂) wherein
X is —CO— or —COO—.

7. A compound and pharmaceutically acceptable salts or stereoisomers thereof according to claim 5 wherein:
m is 1 and
in R of formula (II):
A is a carbon atom,
R¹ is —CH₃,
R² is an hydrogen atom and
R′ is the group —(X—Y—ONO₂) wherein X is —CO— or —COO—.

8. A compound of general formula (I) according to claims 5 to 7, wherein Y is a bivalent radical having the following meaning:
a)
straight or branched C₁-C₁₀ alkylene;
straight or branched C₁-C₁₀ alkylene substituted with one or more —ONO₂; 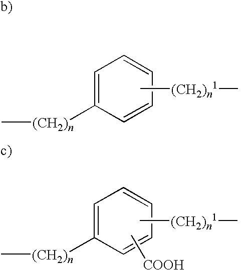
wherein n is an integer from 0 to 5, and n¹ is an integer from 1 to 10; 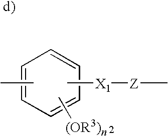
wherein:
X₁=—OCO—;
Z is —(CH₂)ₙ¹— and n¹ is an integer from 1 to 10;
and n² is 1 and R³ is CH₃; 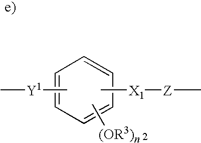
wherein:
Y¹ is —CH₂CH₂—(CH₂)ₙ^{2a} or —CH═CH—(CH₂)ₙ^{2a} wherein n^{2a} is 0 or 1;
X₁ is —OCO—;
Z is —(CH₂)n¹- and n¹ is an integer from 1 to 10;
n² is 1, R³ is CH₃; 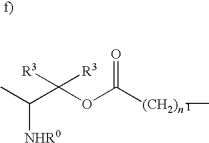
wherein:
n is an integer from 1 to 5;
R³ is H and R⁰ is —COCH₃;
with the proviso that when Y is selected from the bivalent radicals mentioned under b)-f), then the terminal —ONO₂ group is bound to —(CH₂)ₙ¹; 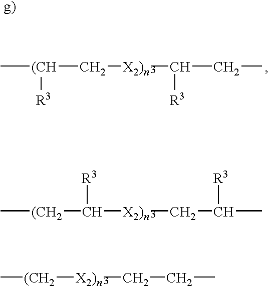
wherein X₂ is —O— or —S—,
n³ is 1 and R³ is H or CH₃; 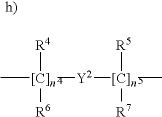
wherein:
n⁴ is an integer from 0 to 5;
n⁵ is an integer from 1 to 5;
R⁴, R⁵, R⁶, R⁷ are H;
wherein the —ONO₂ group is linked to 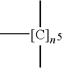
Wherein n⁵ is as defined above;
Y² is selected from

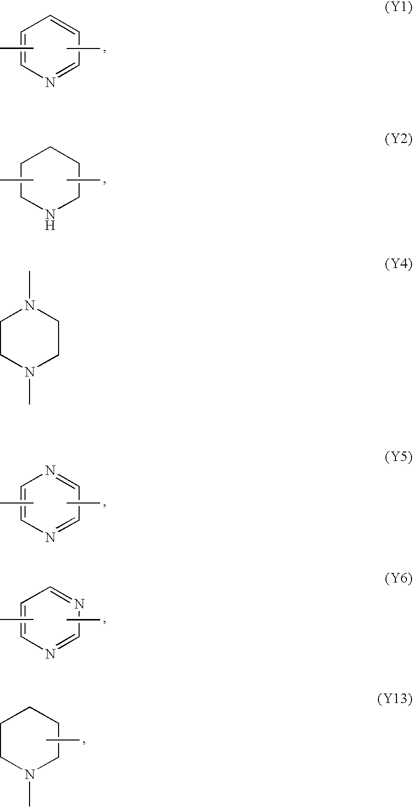

9. A compound according to claim 5 selected from the group consisting of:

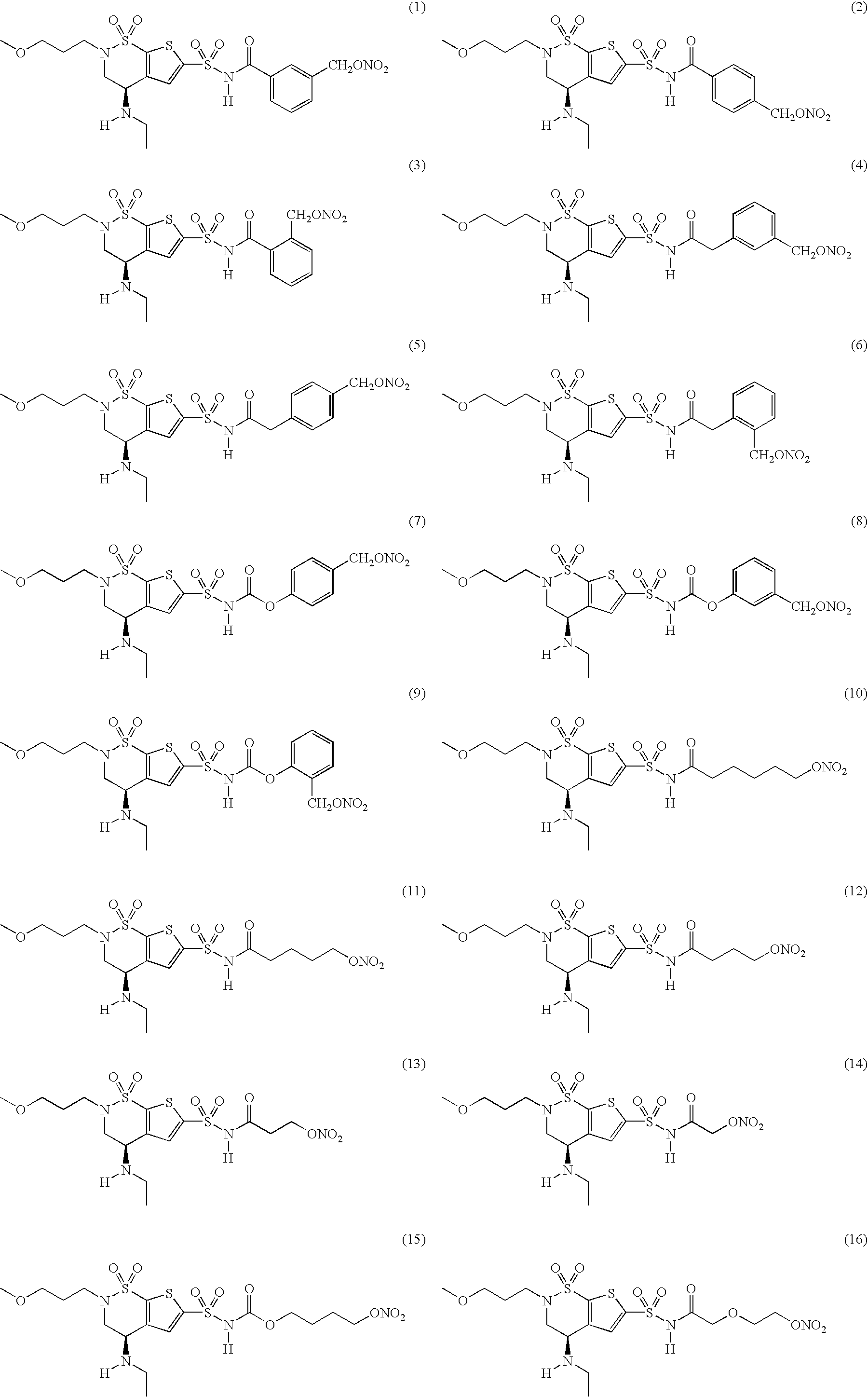

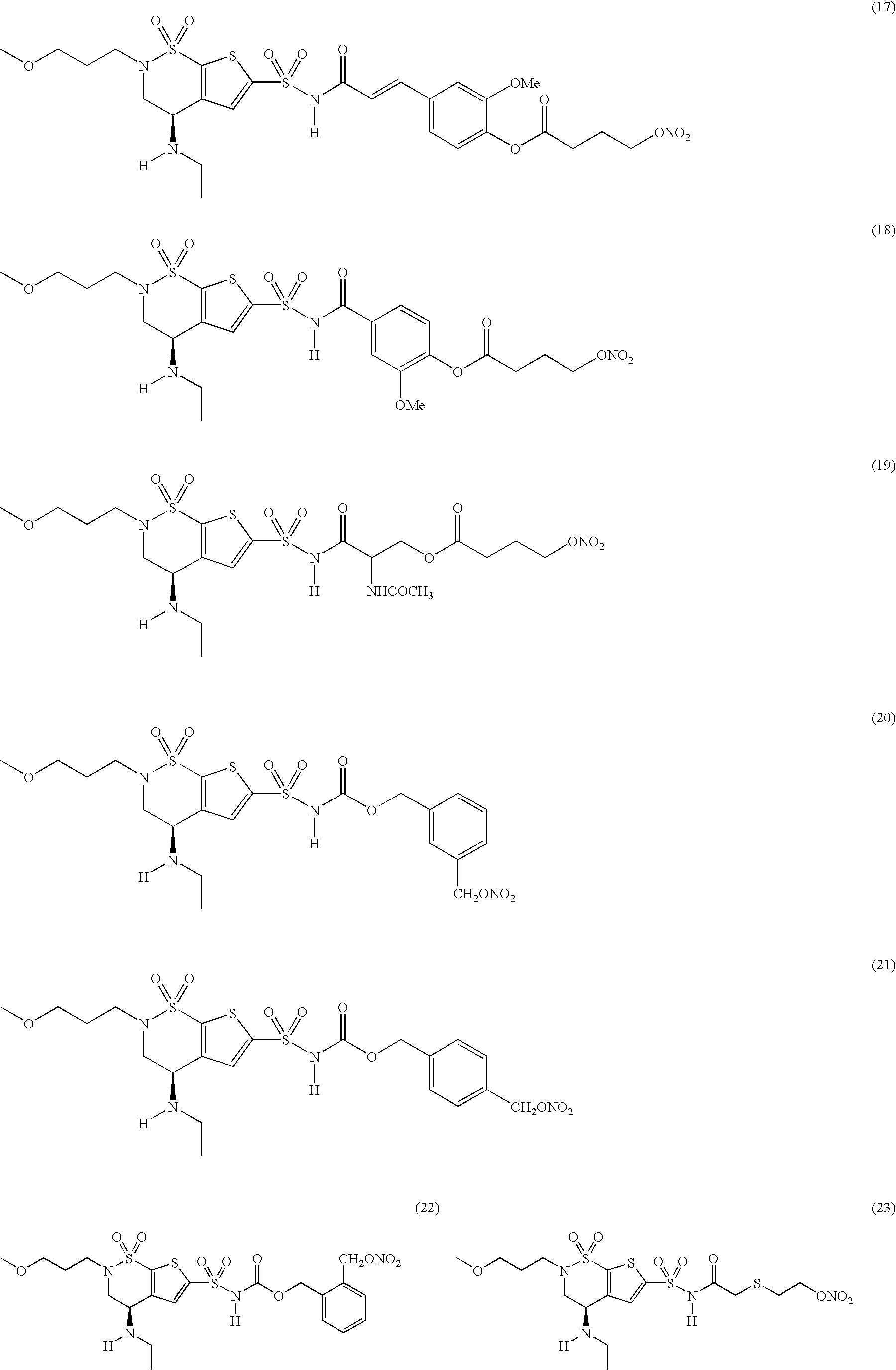

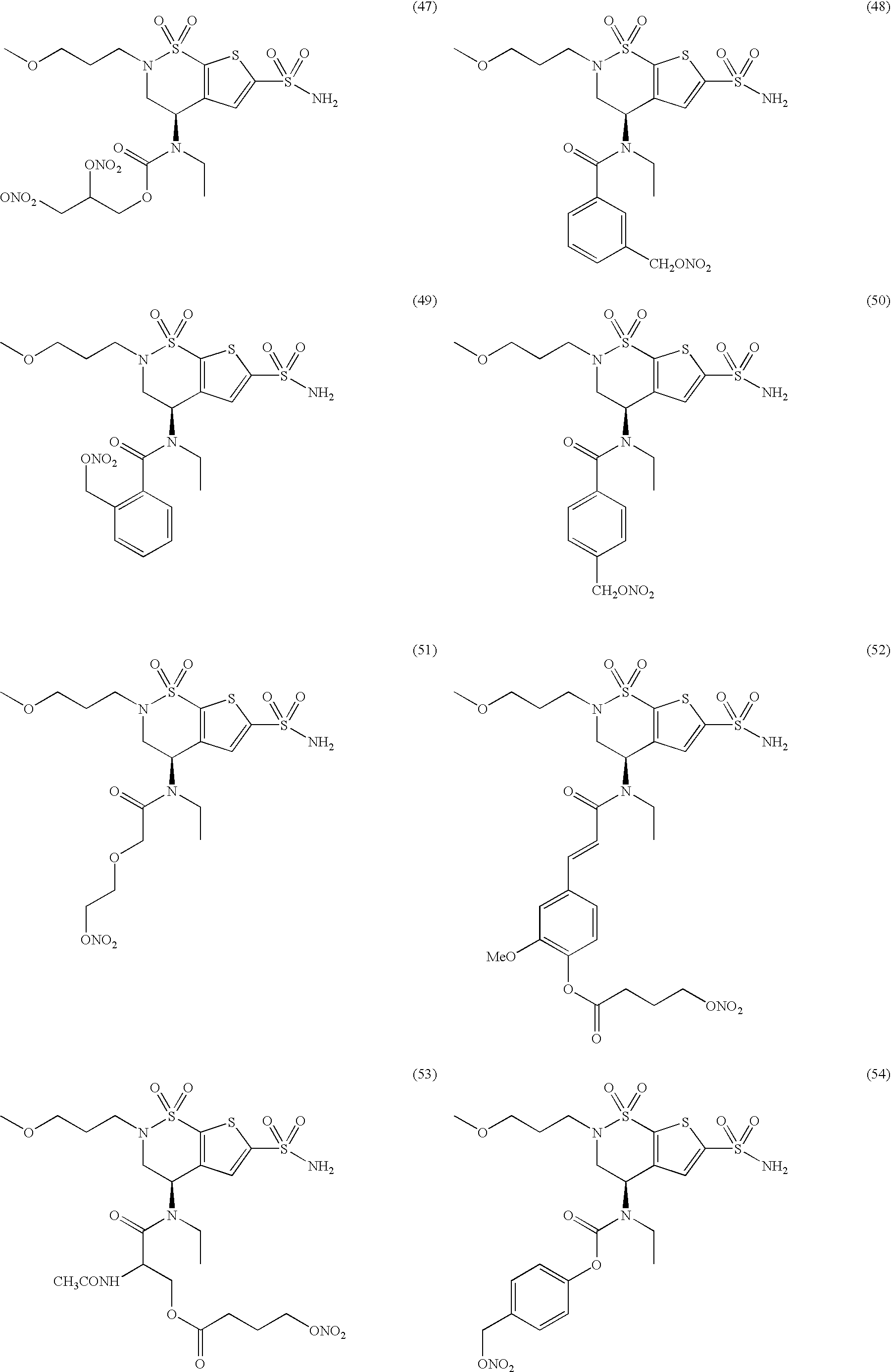

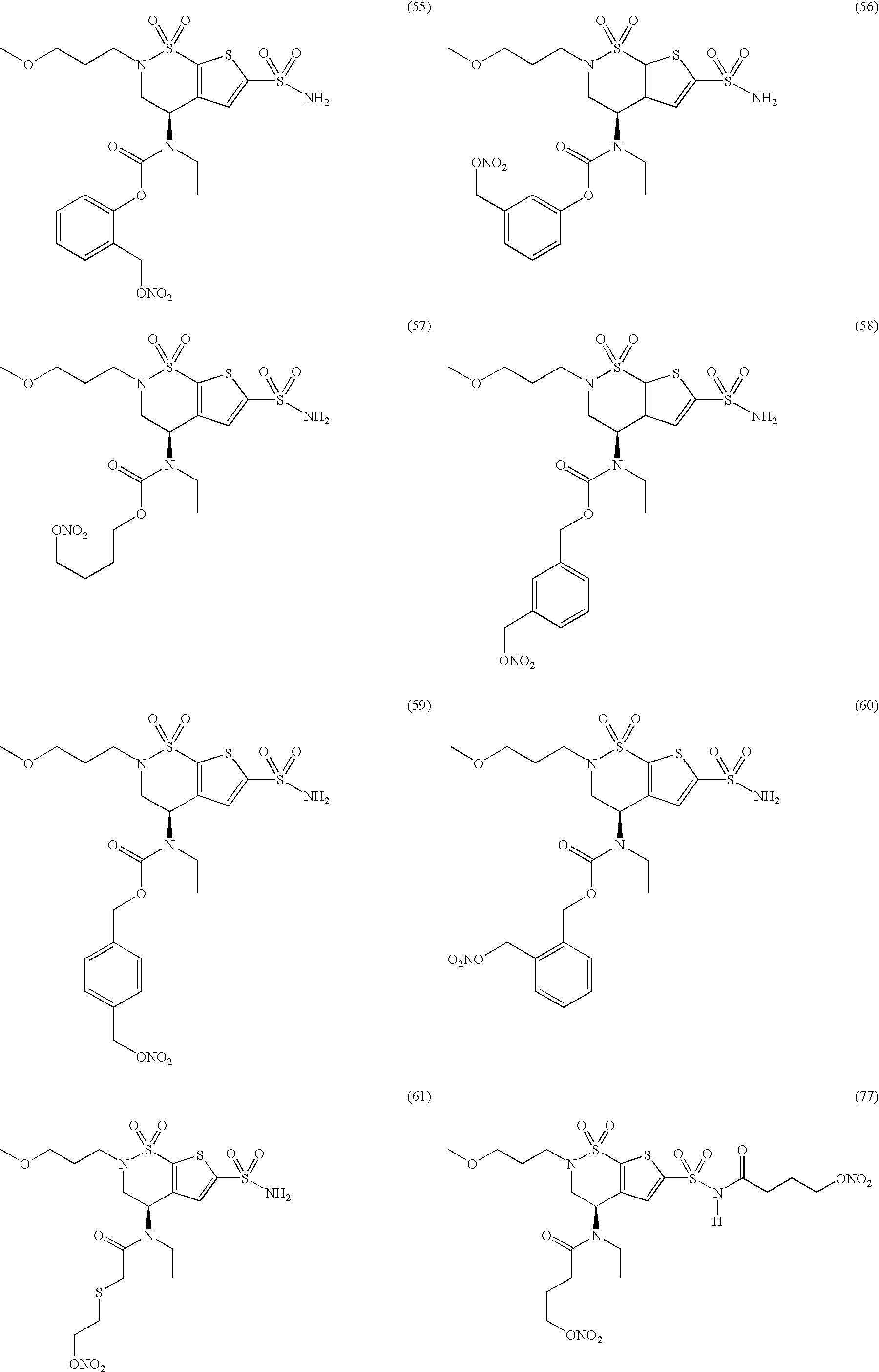

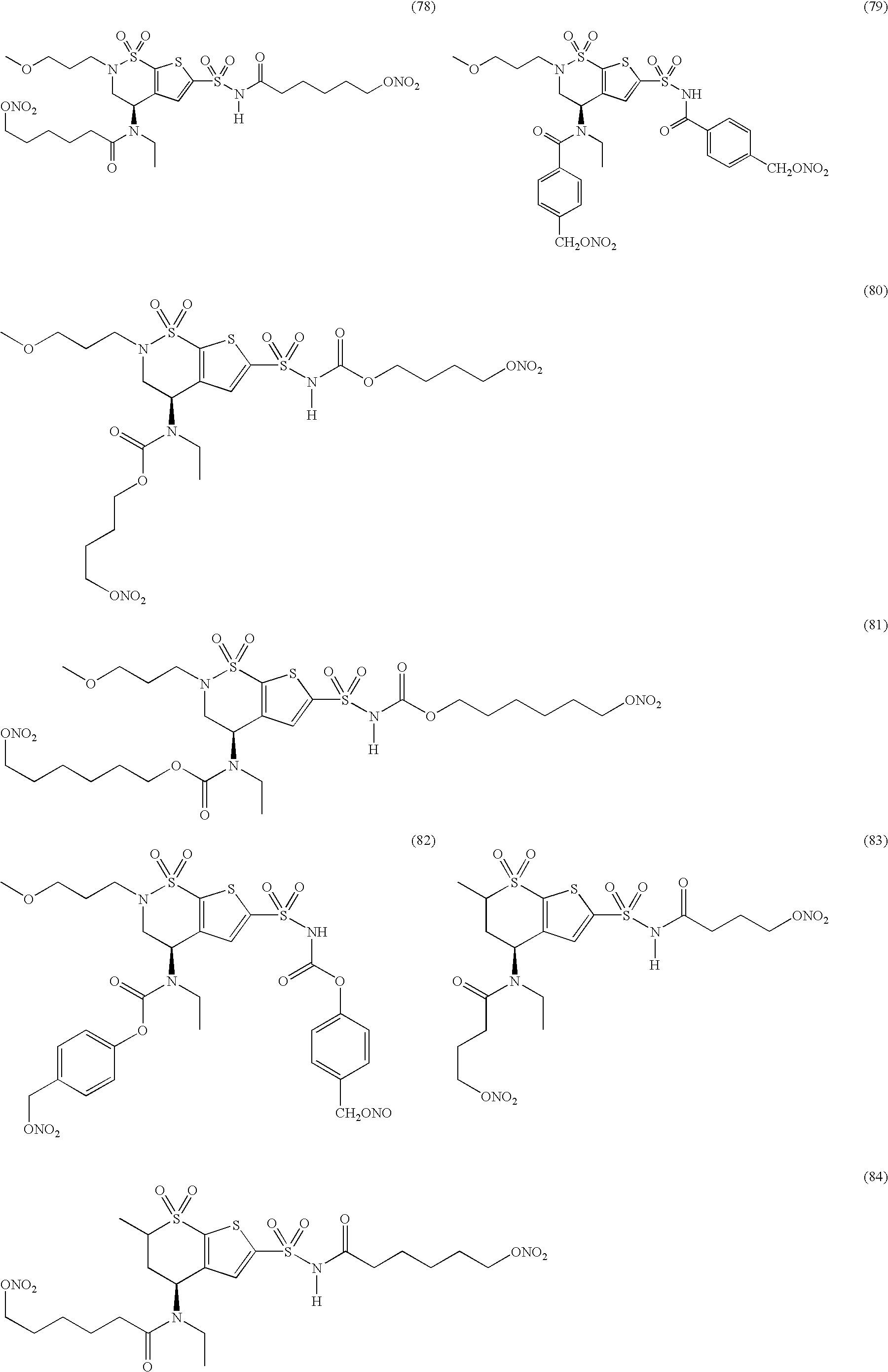

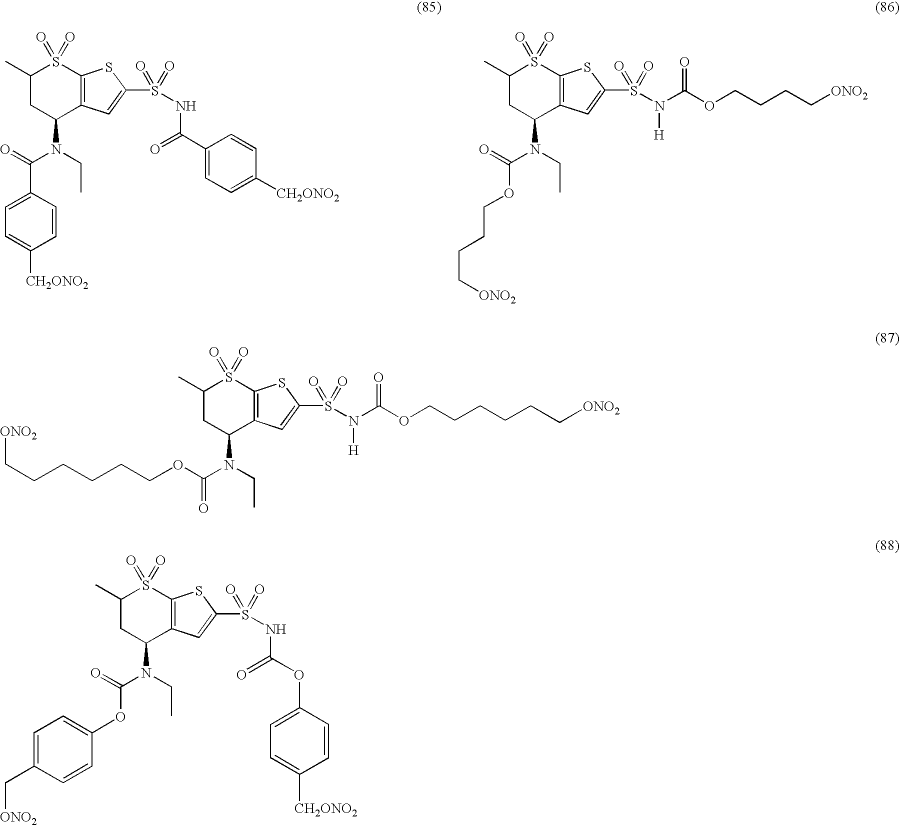

10. A compound according to claim 6 selected from the group consisting of:

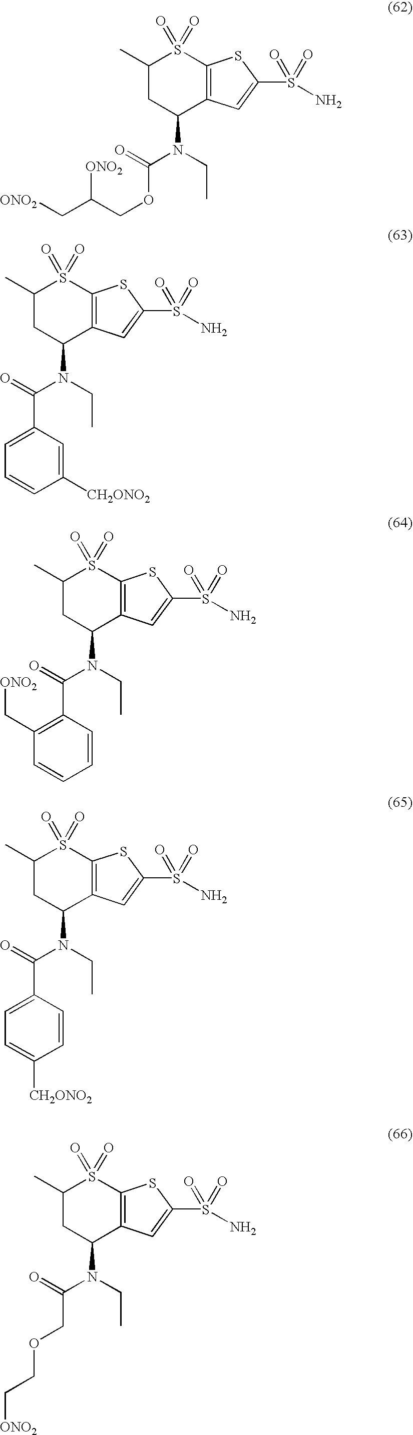

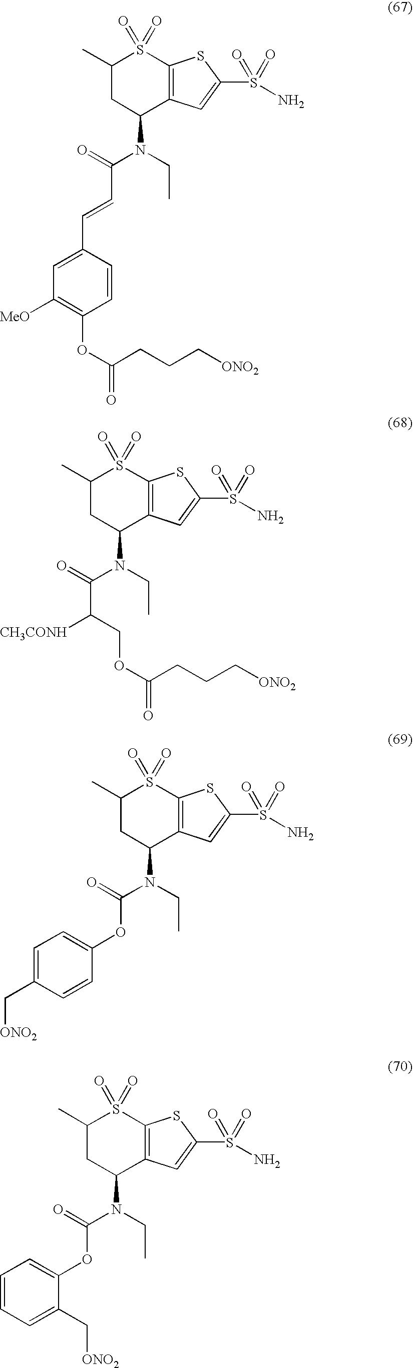

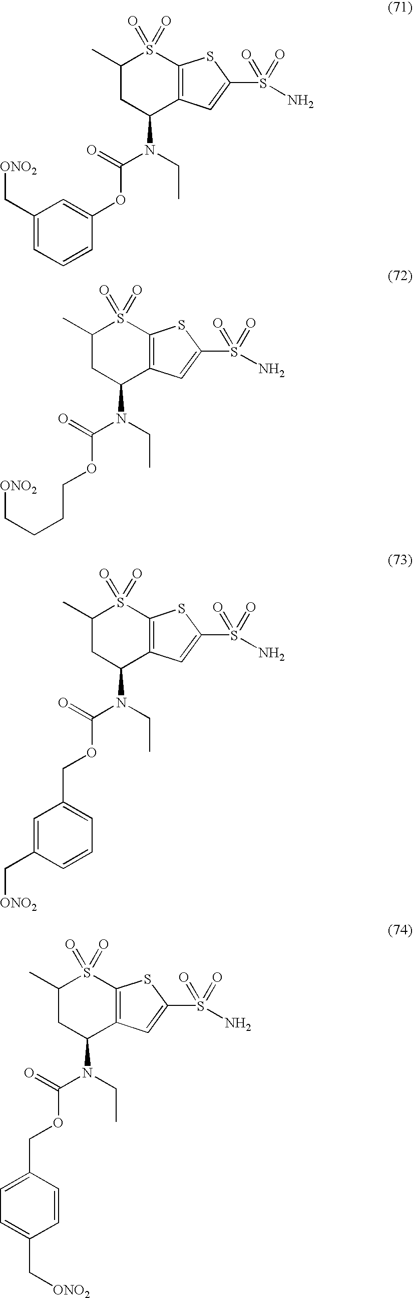

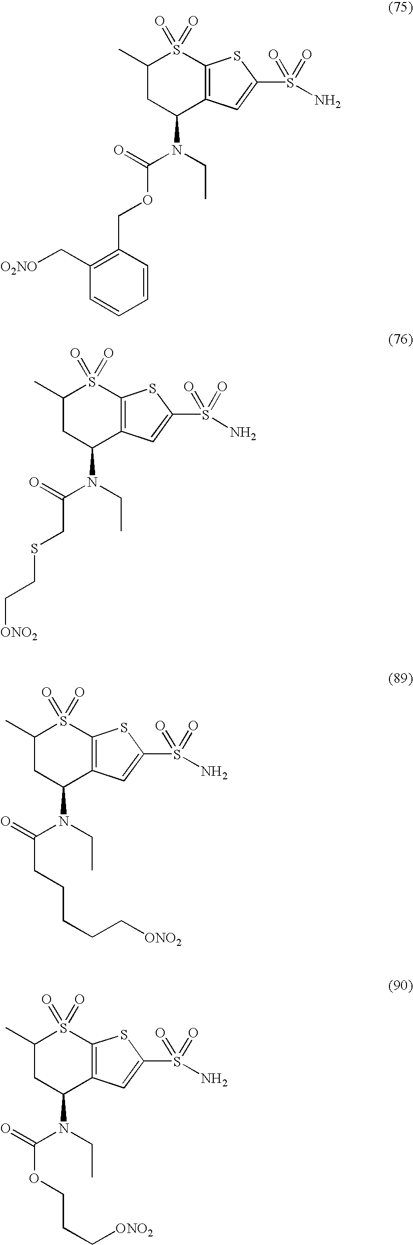

11. A compound according to claim 7 selected from the group consisting of:

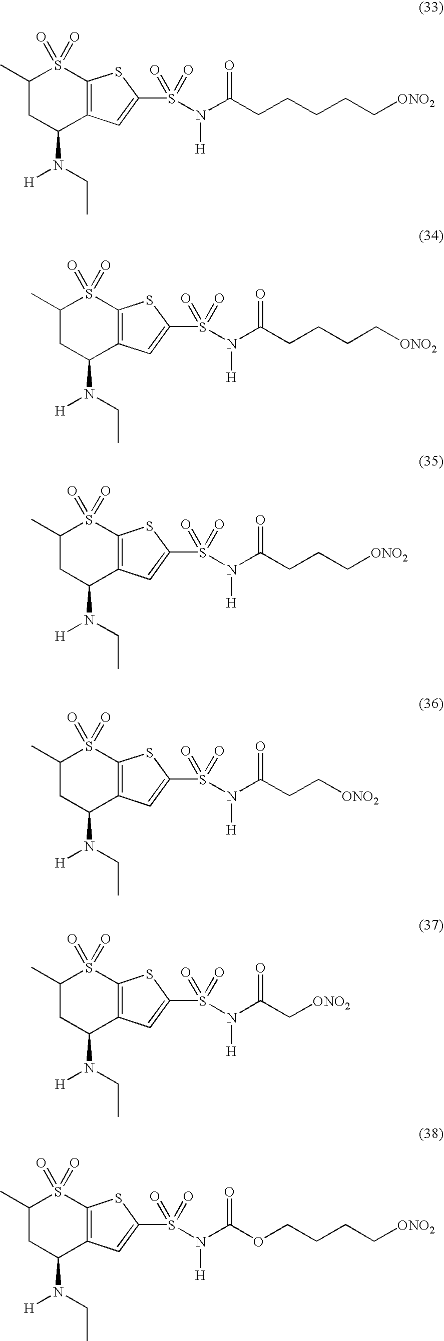

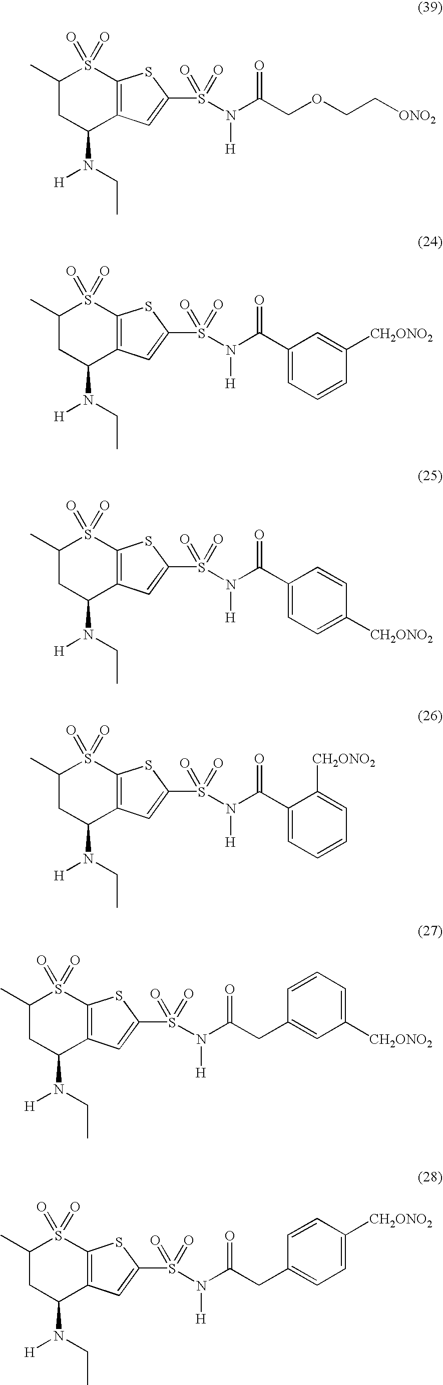

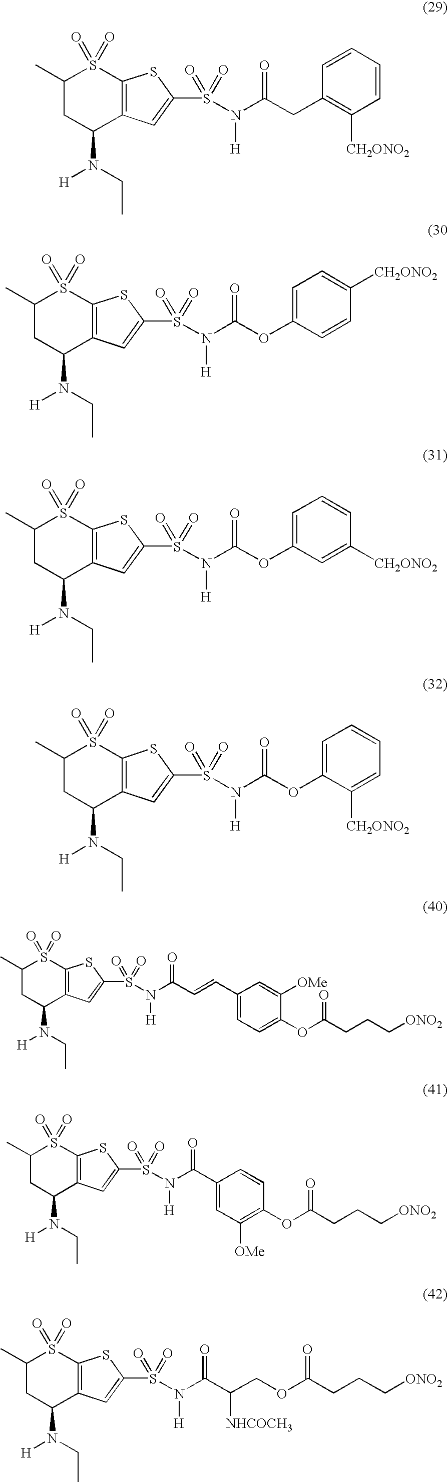

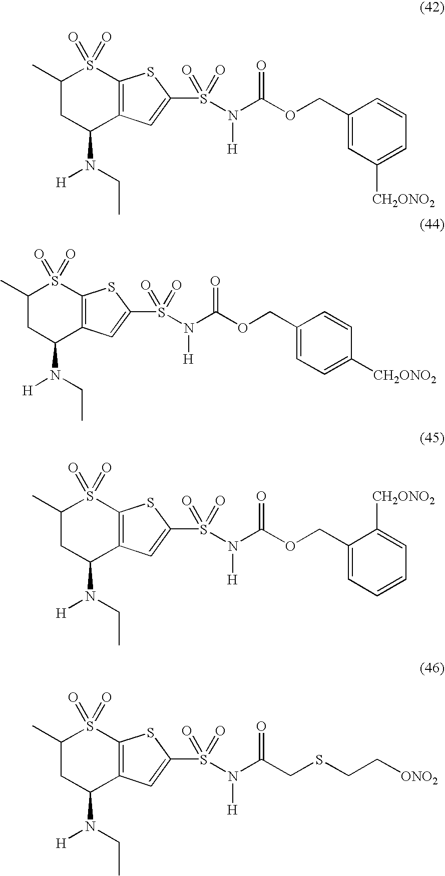

12. A method for the treatment of glaucoma, ocular hypertension, age-related macular degeneration, diabetic macular edema, diabetic retinopathy, hypertensive retinopathy and retinal vasculopathies comprising administering a compound of general formula (I) and/or a salt or stereoisomer thereof according to claim 5.

13. A pharmaceutical composition comprising a pharmaceutically acceptable carrier and a pharmaceutically effective amount of a compound of general formula (I) and/or a salt or stereoisomer thereof as defined in claim 5.

14. A pharmaceutical composition according to claim 13 in a suitable form for the topical administration.

15. A pharmaceutical composition according to claim 13, wherein the compound of general formula (I) is administered as a solution, suspension or emulsion in an ophthalmically acceptable vehicle.

16. A pharmaceutical composition comprising a mixture of a compound of general formula (I) according to claim 5 and (i) a beta-adrenergic antagonists or (ii) a prostaglandin analog or (iii) an a-adrenergic agonist or a nitrooxy derivative thereof.

17. A pharmaceutical composition comprising a mixture of a compound of general formula (I) according to claim 5 and timolol or a nitrooxy derivative thereof.

18. A pharmaceutical composition comprising a mixture of a compound of formula (I) according to claim 5 and latanoprost or a nitrooxy derivative thereof.

19. A pharmaceutical kit for simultaneous, successively or previously administration of a composition according to claim 13 and (i) a beta-adrenergic antagonists or (ii) a prostaglandin analog or (iii) an a-adrenergic agonist or a nitrooxy derivative thereof.

20. A method for the treatment of glaucoma, ocular hypertension, age-related macular degeneration, diabetic macular edema, diabetic retinopathy, hypertensive retinopathy and retinal vasculopathies comprising administering a pharmaceutical composition according to claim 16.
